(19) United States
(12) Patent Application Publication
(10) Pub. No.: US 2024/0269190 A1
(43) Pub. Date: Aug. 15, 2024

(54) CELLULAR COMPOSITIONS FOR TREATING BACK PAIN

(71) Applicants: The United States Government As Represented By The Department of Veterans Affairs, Washington, DC (US); THE TRUSTEES OF THE UNIVERSITY OF PENNSYLVANIA, PHILADELPHIA, PA (US)

(72) Inventors: LACHLAN JAMES SMITH, Philadelphia, PA (US); CHENGHAO ZHANG, Philadelphia, PA (US)

(21) Appl. No.: 18/420,721

(22) Filed: Jan. 23, 2024

Related U.S. Application Data
(60) Provisional application No. 63/481,098, filed on Jan. 23, 2023.

(51) Int. Cl. A61K 35/32

(52) U.S. Cl. CPC A61K 35/32

(57) ABSTRACT

Described are compositions containing isolated mammalian nucleus pulposus cells having an extracellular matrix (ECM)-generating phenotype and methods for making and using the disclosed compositions. The compositions disclosed herein are useful for treating back pain.

### CROSS REFERENCE TO RELATED APPLICATIONS

This application claims the benefit of U.S. Provisional Application No. 63/481,098, filed Jan. 23, 2023. The content of this earlier filed application is hereby incorporated by reference herein in its entirety.

### GOVERNMENT RIGHTS

This invention was made with government support under AR077261, AR077435, P30AR069619 awarded by the National Institutes of Health and I01RX001321 awarded by the Department of Veteran's Affairs. The government has certain rights in the invention.

### TECHNICAL FIELD

Disclosed herein are compositions containing a mammalian nucleus pulposus cells having an extracellular matrix (ECM)-generating phenotype and expressing CD9 and/or CD109 and methods of using the same.

Disclosed herein are compositions containing isolated mammalian nucleus pulposus cells having an extracellular matrix (ECM)-generating phenotype and methods for making and using the disclosed compositions. The compositions disclosed herein are useful for treating back pain.

### BACKGROUND

Intervertebral disc degeneration is a major cause of low back pain, the leading cause of disability worldwide (Global Burden of Disease Study, C. Global, regional, and national incidence, prevalence, and years lived with disability for 301 acute and chronic diseases and injuries in 188 countries, 1990-2013: a systematic analysis for the Global Burden of Disease Study 2013. Lancet. 2015 Aug 22:386(9995): 743-800). Intervertebral disc degeneration is a progressive, inflammation mediated cascade that leads to structural and mechanical failure (Freemont, A. J., Rheumatology (Oxford). 2009 Jan;48(1):5-10). Current treatment approaches for disc degeneration and low back pain are limited to addressing the symptoms but not the cause. In severe cases, patients may undergo surgical procedures such as spinal fusion, which do not maintain or restore native tissue structure or mechanical function (Eck, et al. (2014). J Neurosurg Spine. 21, 42-47). Patients who are not candidates for surgery receive conservative treatments, such as physical therapy and pain killers (P. P. Raj (2008). Pain Pract. 8, 18-44). Of the total patients that present to physicians each year in the U.S. with chronic back pain, about 4 million will have moderate severity degeneration and be neither candidates for surgery nor responsive to conservative therapies (Hart, L. G., et al. (1995). Spine (Phila Pa 1976). 20, 11-19). Emerging cell-based therapies targeting the disc nucleus pulposus (NP), particularly those employing adult stem cells, have shown promise in preclinical studies (Smith, L. J., et al. JOR Spine. 2018 Dec; 1(4):e1036). During development. NP cells uniquely arise from the embryonic notochord (Choi, K. S., et al., Dev Dyn 237(12) (2008) pp. 3953-8; and McCann, M. R., et al., Dis Model Mech 5(1) (2012) pp. 73-82), transitioning from a role focused on tissue patterning through secretion of long-range morphogens, to one more focused on extracellular matrix (ECM) production and maintenance (Peck, S. H., et al. Sci Rep 7(1) (2017) pp 10504; and Dahia, C. L., et al. Spine (Phila Pa 1976) 34(5) (2009) pp 447-55). The mechanisms underlying this transition in NP cell function remain poorly understood. These mechanisms may provide important clues for optimizing adult stem cells for therapeutic, regenerative application. However, efficacy of the cell-based therapies has been limited by the inability of these therapeutic cells to sufficiently mimic the phenotype of native NP cells, including survival in the nutrient poor disc microenvironment and production of a proteoglycan-rich ECM.

There remains a need for cell-based therapies to treat back pain with regenerative NP cells capable of ECM production and maintenance. The present disclosure addresses these needs.

### SUMMARY

In meeting these needs, the present disclosure provides a cellular composition comprising an isolated mammalian nucleus pulposus cell population isolated from other population(s) of nucleus pulposus cells and having an ECM-generating phenotype, and a protectant. In some aspects, the cellular composition comprises a mammalian nucleus pulposus cell population having an ECM-generating phenotype and (i) expressing CD9 or (ii) expressing CD109. In some aspects, the cellular composition comprises a mammalian nucleus pulposus cell population having an ECM-generating phenotype and (i) expressing CD9 and having an anti-CD9 antibody bound to at least a portion of the cells in the population, or (ii) expressing CD109 and having an anti-CD109 antibody bound to at least a portion of the cells in the population. In some aspects, the compositions further comprise mesenchymal stem cells and/or aggrecan.

Also provided are methods of treating a subject. In some aspects, disclosed are methods of treating back pain (e.g., concomitant low back pain) or intervertebral disc degeneration in a subject, The methods include administering one or more of the disclosed cellular compositions to the subject.

Provided are also methods of making the disclosed cellular compositions. The methods of making can comprise combining an isolated mammalian nucleus pulposus cell population, isolated from other population(s) of nucleus pulposus cells, and having an ECM-generating phenotype with a protectant. The methods of making can include isolating the mammalian nucleus pulposus cell population having an ECM-generating phenotype from other population(s) of nucleus pulposus cells and expanding the isolated cells in vitro.

In some aspects, the methods of making the disclosed cellular compositions comprise isolating a mammalian nucleus pulposus cell population that (i) expresses CD9 and/or (ii) expresses CD109.

Disclosed herein are cellular compositions, comprising: a protectant and an isolated mammalian nucleus pulposus cells, wherein the isolated mammalian nucleus pulposus cells have an extracellular matrix (ECM)-generating phenotype.

Disclosed herein are cellular compositions comprising: a protectant and a population of mammalian nucleus pulposus cells having an extracellular matrix (ECM)-generating phenotype, wherein the mammalian nucleus pulposus cells (i) express CD9 and have an anti-CD9 antibody bound to at least a portion of the cells, and/or (ii) express CD109 and have an anti-CD109 antibody bound to at least a portion of the cells.

Disclosed herein are methods of treating a subject, the method comprising administering to the subject a cellular composition comprising: a protectant and isolated mammalian nucleus pulposus cells, wherein the isolated mammalian nucleus of pulposus cells of nucleus pulposus cells have an extracellular matrix (ECM)-generating phenotype.

Disclosed herein are methods of treating a subject, the method comprising administering to the subject a cellular composition comprising: a protectant and a population of mammalian nucleus pulposus cells having an extracellular matrix (ECM)-generating phenotype wherein the mammalian nucleus pulposus cells (i) express CD9 and have an anti-CD9 antibody bound to at least a portion of the cells, and/or (ii) express CD109 and have an anti-CD109 antibody bound to at least a portion of the cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The summary, as well as the following detailed description, is further understood when read in conjunction with the appended drawings. For the purpose of illustrating the disclosed compositions and methods there are shown in the drawings exemplary embodiments of compositions and methods; however, these should not be limited to the specific embodiments disclosed. In the drawings:

FIG. 1A is a Uniform Manifold Approximation and Projection (UMAP) plot showing clustering of two distinct populations of notochord-derived mouse NP cells. FIG. 1B is a graph with differentiation trajectory analysis showing the pseudo-temporal transition from cluster 1 to cluster 2 cells.

FIGS. 2A-2H are UMAP plots showing relative expression of mouse NP marker genes KRT19, KRT18, and T (FIGS. 2A-2C, respectively), ECM marker genes ACAN, COL2A1, and COL6A1 (FIGS. 2D-2F, respectively), and cell surface marker genes CD9 and CD109 (FIGS. 2G and 2H, respectively) in each cell cluster.

FIGS. 3A-3C are violin plots from mouse NP cell scRNA-Seq analysis showing that cells in cluster 2 highly express aggrecan (FIG. 3A), the most important extracellular matrix constituent of the NP. Cells in cluster 2 concomitantly express the surface markers CD9 (FIG. 3B) and CD109 (FIG. 3C).

FIGS. 4A-4F are graphs showing mRNA expression levels for markers CD9 (FIG. 4A), CD109 (FIG. 4B), CD24a (FIG. 4C), CD34 (FIG. 4D), CD44 (FIG. 4E), and Nanog (FIG. 4F) in the mouse NP cells for cluster 1, and cluster 2. The graphs show absence of increased expression of CD24a, CD34, CD44, and Nanog in mouse NP cells for cluster 2.

FIGS. 5A-5E are images showing TdTomato+ cells in the NPs of discs from Shh-cre:R26R:tdTomato mice aged 0 to 60 days (top panels). Alcian blue/picrosirius red (ABPR)-stained sections showing progressive accumulation of GAG-rich ECM in the peripheral regions of the NP with increasing age (middle panels). Immunofluorescent staining showing CD9-positive cells confined to the peripheral regions of the NP (bottom panel). FIG. 5A shows mice aged 0 days. FIG. 5B shows mice aged 7 days. FIG. 5C shows mice aged 14 days. FIG. 5D shows mice aged 30 days. FIG. 5E shows mice aged 60 days. Scale=100 μm.

FIG. 6A is a table listing percent viability of isolated goat cells (NP cells or NP cells plus MSCs) from each condition prior to scRNA-Seq. FIGS. 6B and 6C are representative images showing in situ live (green, 1) and dead (red, 2) staining of cells for organ culture groups (scale=100 μm).

FIGS. 7A-7C are UMAP plots showing the presence of six distinct cell clusters (1-6) for each of the three experimental conditions. FIG. 7D is a graph showing how the percentage of cells in each cluster varied between experimental conditions.

FIGS. 8A-8F are violin plots showing relative expression of ECM and inflammatory/cell stress genes (ACAN, FIG. 8A; COL2A1, FIG. 8B; COL6A3, FIG. 8C; JUN, FIG. 8D; NFKBIA, FIG. 8E; AND PTGS2, FIG. 8F) in each cluster (1-6). FIGS. 8G-8I are UMAP plots showing relative expression of ACAN genes in each cluster (1-6) for each experimental group. FIG. 8G shows freshly isolated NP cells; FIG. 8H shows NP cells after organ culture; and FIG. 8I shows NP cells and MSCs after organ culture.

FIGS. 9A-9C are violin plots from goat NP cell scRNA-Seq analysis showing that cells in cluster 2 highly express aggrecan, the most important extracellular matrix constituent of the NP. Furthermore, cells in cluster 2 concomitantly express the surface markers CD9 and CD109. These results parallel findings for the mouse NP. FIG. 9A shows expression level of ACAN; FIG. 9B shows expression level of CD9; and FIG. 9C shows expression level of CD109.

FIGS. 10A-10D are graphs showing mRNA expression levels for markers CD109 (FIG. 10A), CD9 (FIG. 10B), CD24 (FIG. 10C), and CD44 (FIG. 10D) in the goat NP cells for clusters 1-6. The graphs show absence of increased expression of CD24 and CD44 in goat NP cells for cluster 2.

FIG. 11 shows co-localization of aggrecan and CD-9 expression in the postnatal mouse NP.

FIG. 12 shows that the majority of adult, healthy human and goat NP cells express CD-9.

FIG. 13A shows CD-9 positive cells in the NP of adult healthy and degenerate goat disc, and FIG. 13B shows quantification of CD-9 positive cells in the healthy and degenerate goat disc.

FIG. 14 shows the flow cytometry analysis of nucleus pulposus cells isolated from 9 week old porcine NPs demonstrating that 39% of cells express Cd9+ corresponding to mature, extracellular matrix producing cells.

FIG. 15A shows in situ fluorescence imaging of sections from Shh/tdTomato mouse lumbar spines showing tdTomato+ cells (red) localized to the NP throughout postnatal growth. Krt19 immunofluorescence (green) on the same sections was used to confirm NP cell identity. Blue: cell nuclei (DAPI); mid-sagittal sections; scale=100 μm. FIG. 15B shows representative plots from fluorescence activate cell sorting used to enrich tdTomato+ cells.

FIG. 15C shows a UMAP plot of scRNA-seq results of cells isolated from P30 mice showing clustering of 15 different cell populations. FIG. 15D depicts dot plots showing cluster-specific expression of NP cell markers, hematopoietic, endothelial and blood cell markers, and mesenchymal cell markers. H=Hematopoietic; Bl=blood; C=Cartilage; NP=Nucleus Pulposus; E=Endothelial; Bn=Bone; and F=Fibrocartilage.

FIG. 16A shows a UMAP plot of scRNA-seq results for NP cells, identifying two distinct cell subpopulations. FIG. 16B shows the differentiation trajectory analysis demonstrated that NP cells in clusters 1 and 2 aligned along a pseudo timeline; and FIG. 16C shows RNA velocity analysis showed that the direction of differentiation was predominantly from cluster 1 to cluster 2. Based on these findings, cluster 1 and 2 cells were denoted “progenitor” and “mature” NP cells, respectively. FIG. 16D shows UMAP plots for NP marker genes, showing that Krt8, Krt18, Krt19 and T were expressed across both progenitor and mature NP cells. FIG. 16E shows UMAP plots and FIG. 16F shows violin plots showing significantly (*) higher expression of NP-specific ECM genes in mature NP cells (log2 fold change).

FIGS. 17A-G show pathway analysis. FIG. 17A shows the top 20 GO terms significantly enriched in mature versus progenitor NP cells. FIG. 17B shows the pathways significantly enriched in mature versus progenitor NP cells identified by KEGG analysis. Heatmaps showing differentially expressed genes in the ECM-receptor interactions (FIG. 17C); FIG. 17D shows PI3kT-Akt; FIG. 17E shows protein digestion and absorption; FIG. 17F shows focal adhesion; and FIG. 17G shows TGF-β signaling pathways respectively, between mature and progenitor NP cells.

FIG. 18A shows gene network analysis for 5 pathways enriched in mature versus progenitor NP cells. FIG. 18B shows secreted signaling interactions between NP cell populations identified using CellChat.

FIGS. 19A-E depict UMAP and violin plots showing significantly (*) higher expression of the surface markers Cd109 (FIG. 19A) and Cd9 (FIG. 19B) in mature versus progenitor NP cells (log2 fold change). FIG. 19C shows in situ fluorescence imaging of tdTomato+ cells (red), and corresponding immunofluorescence imaging of Cd109+ (green) and Cd9+ (white) cells in the NPs of mouse lumbar spines during postnatal growth. Cd9+ cells were localized to the NP periphery, with relative numbers increasing with postnatal age. Blue (DAPI)=cell nuclei; Midsagittal sections; 100 μm. FIG. 19D shows flow cytometry analysis showing relative increases in the number of tdTomato/Krt19/Cd9+ positive cells with increasing postnatal age. FIG. 19E depicts representative sections showing progressive accumulation of glycosaminoglycan-rich ECM at the NP periphery with increasing postnatal age. Alcian blue and picrosirius red staining; mid-sagittal sections; scale=100 μm.

FIG. 20A shows representative histological sections of healthy and degenerate goat lumbar discs. Alcian blue and picrosirius red-staining; mid-sagittal sections; scale=2 mm. FIG. 20B shows semi-quantitative grading of healthy and degenerate goat discs. *p<0.05 vs healthy; N=3-4; Students t-test. FIG. 20C shows representative immunostaining of Cd9+ cells in the NPs of healthy and degenerate goat discs. Mid-sagittal sections; scale=100 μm (left) and 20 μm (right). FIG. 20D shows quantification of the relative numbers of Cd9+ cells in the NPs of healthy and degenerate goat discs. *p<0.05 vs healthy; N=3-4; Students t-test. FIG. 20E shows flow cytometry analysis showing the relative number of Cd9+ positive cells in the healthy, adult goat NP.

FIG. 21A is a schematic representations of the transition from progenitor to mature NP cells, which is characterized by increased ECM-receptor interaction, PI3kT-Akt, protein digestion and absorption, focal adhesion and TGF-β signaling, and expression of aggrecan, collagens II and VI, Sox9 and Cd9. FIG. 21B is a schematic representation of emergent mature NP cells during postnatal growth with concomitant peripheral ECM deposition.

FIG. 22A shows a UMAP of NP cells showing cell cycle state. FIG. 22B depicts violin plots showing relative expression of cell proliferation markers in early and late stage NP cells.

### DETAILED DESCRIPTION

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

When values are expressed as approximations, by use of the antecedent “about,” it will be understood that the particular value forms another embodiment. Reference to a particular numerical value includes at least that particular value, unless the context clearly dictates otherwise.

“Substantial” refers to a degree of similarity, difference, increase, or decrease, as in a comparison to a known value. Substantial can include at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% similarity, difference, increase, or decrease, as in a comparison to a known value.

The terms “treating” or “treatment” refer to any success or indicia of success in the attenuation or amelioration of intervertebral disc degeneration and/or back pain, including any objective or subjective parameter such as abatement, diminishing of one or more symptoms of intervertebral disc degeneration and/or back pain, or making the injury, pathology, or condition more tolerable to the subject, improving the subject's physical well-being, or prolonging the length of survival. The treatment or amelioration of the one or more symptoms can be based on objective or subjective parameters; including the results of a physical examination, laboratory test(s), non-invasive imaging test(s), and/or self-reporting by the subject.

The terms “effective amount” and “therapeutically effective amount” are used interchangeably herein and refer to an amount of the cell composition effective to achieve a particular biological or therapeutic result such as, but not limited to, amelioration of one or more symptoms of back pain. A therapeutically effective amount of a composition may vary according to factors such as the disease state, age, sex, body surface area, and body weight of the individual, and the ability of the composition to elicit a desired response in the individual. Such results may include, but are not limited to, the treatment of back pain, as determined by any means suitable in the art.

The term “subject” as used herein is intended to mean a mammal. The methods described herein are applicable to human and nonhuman animals, although preferably used with pets and humans, and most preferably with humans. “Subject” and “patient” are used interchangeably herein. In some embodiments, the subject is human.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. The materials, methods, and examples disclosed herein are illustrative only and not intended to be limiting.

It is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise. The term “about” as used herein when referring to a measurable value such as an amount, a temporal duration, and the like, is meant to encompass variations of ±10%, ±5%, ±1%, or ±0.1% from the specified value, as such variations are appropriate to perform the disclosed methods.

As used herein, approximating language may be applied to modify any quantitative representation that may vary without resulting in a change in the basic function to which it is related. Accordingly, a value modified by “about” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only.

As used in this specification and the appended claims, the singular forms “a,” “an,” and “the” include plural referents unless the content clearly dictates otherwise. Thus, for example, reference to “a cell” includes a combination of two or more cells, and the like.

As used herein, the term “isolated,” in a context of cells, refers to cells that are separated, enriched, or purified from a mix of cells. Some or all of the isolated cells may contain a label used for isolation. Suitable labels include cell-surface marker-specific antibodies, antibody binding fragments, and any derivatives thereof.

As used herein, the phrase “extracellular matrix (ECM)-generating phenotype” refers to a phenotype of cells capable of producing at least one, at least two, at least three, or more components of nucleus pulposus ECM.

The term “an extracellular matrix (ECM)-generating” refers to “maintaining the healthy (normal) condition of the ECM”, which includes stabilized level of transcription and/or translation of aggrecan, and/or prohibited or delayed degradation process of aggrecan.

Intervertebral disc degeneration is a leading cause of chronic low back pain. Cell-based strategies that seek to treat disc degeneration by regenerating the central nucleus pulposus hold significant promise, but challenges remain. One of these challenges is the inability of therapeutic cells to effectively mimic the performance of native nucleus pulposus cells, which are different amongst skeletal cell types in that they arise from the embryonic notochord. Described herein is the use of single cell RNA sequencing to demonstrate emergent heterogeneity amongst notochord-derived nucleus pulposus cells in the postnatal mouse disc. Specifically, the existence of progenitor and mature nucleus pulposus cells, corresponding to notochordal and chondrocyte-like cells, respectively, was established. Mature nucleus pulposus cells exhibited significantly higher expression levels of extracellular matrix genes including aggrecan, and collagens II and VI, along with elevated TGF-β and PI3K-Akt signaling. Additionally, CD9 was identified as a surface marker of mature nucleus pulposus cells, and it was demonstrated that these cells were localized to the nucleus pulposus periphery, increased in numbers with increasing postnatal age, and co-localized with emerging glycosaminoglycan-rich matrix. Finally, a goat model was used to show that the CD9+ nucleus pulposus cell numbers decrease with moderate severity disc degeneration, demonstrating that these cells are associated with maintenance of the healthy nucleus pulposus extracellular matrix.

Lumbar intervertebral disc degeneration is strongly implicated as a cause of low back pain, the leading cause of disability worldwide (Dieleman, J L, et al. JAMA 2016; 316:2627-2646; Freemont, A J. Rheumatology (Oxford) 2009; 48:5-10; Global Burden of Disease Study, Lancet 2015; 386:743-800; and Raj, P P. Pain Pract 2008; 8:18-44). The discs are the partially-movable joints of the spine, and each is comprised of three main substructures: a central, proteoglycan-rich nucleus pulposus (NP); a peripheral, fibrocartilaginous annulus fibrosus (AF) with a highly ordered, cross-ply lamellar structure; and superiorly and inferiorly two cartilaginous end plates that interface with the adjacent vertebrae (Smith, L J, et al. Dis Model Mech 2011; 4:31-41). These three structures act together to facilitate the even distribution of compressive loads between the vertebrae, and complex mobility of the intervertebral joint (Smith, L J, et al. Dis Model Mech 2011; 4:31-41). Disc degeneration is a slowly progressing, cell-mediated cascade that is closely linked to aging, and which ultimately leads to structural and functional derangement of the entire intervertebral joint (Freemont, A J. Rheumatology (Oxford) 2009; 48:5-10; and Raj, P P. Pain Pract 2008; 8:18-44). Current treatments for disc degeneration and associated low back pain, including both conservative approaches (such as pain medication and physical therapy) and surgery (such as spinal fusion), target symptoms but fail to restore healthy disc structure and function, and often have poor long-term efficacy (Raj, P P. Pain Pract 2008; 8:18-44; Eck, J C, et al. J Neurosurg Spine 2014; 21:42-47; Hart, L G, et al. Spine (Phila Pa 1976) 1995; 20:11-19; and Zhang, C, et al. Clin Spine Surg 2016; 29:21-29).

Degeneration manifests initially in the NP, where an inflammation-mediated reduction in proteoglycan content and hydration compromises resistance to compressive loads (Raj, P P. Pain Pract 2008; 8:18-44; Smith, L J, et al. Dis Model Mech 2011; 4:31-41; Le Maitre, et al. Biochem Soc Trans 2007; 35:652-655; and Urban, J P, and Roberts, S. Arthritis Res Ther 2003; 5:120-130). Cell-based strategies to treat disc degeneration by regenerating NP tissue hold significant promise, but important challenges remain (Smith, L J, et al. JOR Spine 2018; 1:e1036; and Loibl, M, et al. JOR Spine 2019; 2:e1043). One of these is the inability of therapeutic cells to effectively mimic the performance of native NP cells, which exhibit the ability to survive in the oxygen and nutrient-poor disc microenvironment whilst simultaneously secreting large quantities of proteoglycan-rich extracellular matrix (ECM) (Smith, L J, et al. Dis Model Mech 2011; 4:31-41). Amongst skeletal cell types, NP cells arise from the embryonic notochord, a midline structure that serves as both a source of long-range morphogens to guide patterning of the axial skeleton, and as the structure that physically gives rise to the discrete NPs themselves (Choi, K S, et al. Dev Dyn 2008; 237:3953-3958; Lawson, L, and Harfe, B D. Curr Osteoporos Rep 2015; 13:336-341; McCann, M R, et al. Dis Model Mech 2012; 5:73-82; and Stemple, D L. Development 2005; 132:2503-2512). In humans, the rapid and complete disappearance of large, vacuolated, notochordal-like NP cells by skeletal maturity, and their replacement by smaller cells that resemble articular cartilage chondrocytes, is thought to contribute the onset of disc degeneration later in life (Smith, L J, et al. Dis Model Mech 2011; 4:31-41; Cappello, R, et al. Spine (Phila Pa 1976) 2006; 31:873-882; discussion 883; and Hunter, C J, et al. J Anat 2004; 205:357-362). In contrast, discs in other species such as mice, while exhibiting similarities to human discs with respect to development, geometry, composition and biomechanical properties ((Smith, L J, et al. Dis Model Mech 2011; 4:31-41; Beckstein, J C, et al. Spine (Phila Pa 1976) 2008; 33:E166-173; and O'Connell, G D, et al. Spine (Phila Pa 1976) 2007; 32:328-333) retain significant numbers of notochordal-like NP cells throughout adulthood and experience milder age-associated disc degeneration compared to humans (Hunter, C J, et al. J Anat 2004; 205:357-362; Tang, S N, et al. Front Pain Res (Lausanne) 2022; 3:894651; and Mohanty, S, et al. Aging Cell 2019; 18:e13006). Due to this retention of notochordal-like NP cells, mice represent an excellent model with which to study dynamic cell heterogeneity within the postnatal NP. Elucidating the molecular mechanisms underlying the phenotypic transition from notochordal to chondrocyte-like NP cells is important to inform the development of more effective stem cell-based therapeutics, by establishing the characteristics for de novo elaboration of the aggrecan-rich NP ECM (Choi, H, et al. Curr Stem Cell Res Ther 2015; 10:307-316).

Previously, bulk RNA-sequencing was used to demonstrate that the function of notochord-derived NP cells transitions from one focused on embryonic patterning to one focused on growth and ECM deposition (Peck, S H, et al. Scientific reports 2017; 7:10504). It was thought that these bulk gene expression changes are due to emergent NP cell subpopulations that drive deposition of the proteoglycan-rich ECM necessary to sustain disc mechanical function in adulthood. To test this, as described herein, single cell RNA sequencing (scRNA-seq) was used and an emergent NP cell subpopulation was identified in the postnatal mouse disc that exhibits high expression of NP-specific ECM genes and the surface marker CD9. Additionally, a goat model was used to show that the relative number of these CD9+ cells decreases significantly coincident with disc degeneration, supporting their association with the maintenance of the healthy NP ECM.

### Compositions

Provided are cellular compositions comprising an isolated mammalian nucleus pulposus cell population having an extracellular matrix (ECM)-generating phenotype and a protectant. The isolated mammalian nucleus pulposus cell population having an extracellular matrix (ECM)-generating phenotype can be isolated from other population(s) of nucleus pulposus cells. In some aspects of the cellular compositions, at least 90% of cells in the isolated mammalian nucleus pulposus cell population express CD9. In some aspects of the cellular compositions, at least 90% of cells in the isolated mammalian nucleus pulposus cell population express CD109. For example, the cellular compositions can include an isolated mammalian nucleus pulposus cell population where at least about 90% of cells express CD9, CD109, or both CD9 and CD109. In some aspects, the cellular compositions can further comprise mesenchymal stem cells and/or aggrecan.

Disclosed are cellular compositions, comprising: a protectant, and isolated mammalian nucleus pulposus cells, wherein the isolated mammalian nucleus of pulposus cells of nucleus pulposus cells have an extracellular matrix (ECM)-generating phenotype.

In some aspects, between about 90% and 100%, between about 90% and about 99%, between about 90% and about 98%, between about 90% and about 97%, between about 90% and about 96%, between about 90% and about 95%, between about 90% and about 94%, or between about 90% and about 93%, of cells in the isolated mammalian nucleus pulposus cell population express CD9. In some aspects, at least about 90%, at least about 91%, at least about 92%, at least about 93%, at least about 94%, at least about 95%, at least about 96%, at least about 97%, at least about 98%, at least about 99%, or about 100% of cells in the isolated mammalian nucleus pulposus cell population express CD9.

In some aspects, between about 90% and 100%, between about 90% and about 99%, between about 90% and about 98%, between about 90% and about 97%, between about 90% and about 96%, between about 90% and about 95%, between about 90% and about 94%, between about 90% and about 93%, of cells in the isolated mammalian nucleus pulposus cell population express CD109. In some aspects, at least about 90%, at least about 91%, at least about 92%, at least about 93%, at least about 94%, at least about 95%, at least about 96%, at least about 97%, at least about 98%, at least about 99%, or about 100% of cells in the isolated mammalian nucleus pulposus cell population express CD109.

In some aspects, between about 90% and 100%, between about 90% and about 99%, between about 90% and about 98%, between about 90% and about 97%, between about 90% and about 96%, between about 90% and about 95%, between about 90% and about 94%, between about 90% and about 93%, of cells in the isolated mammalian nucleus pulposus cell population express CD9 and CD109. In some aspects, at least about 90%, at least about 91%, at least about 92%, at least about 93%, at least about 94%, at least about 95%, at least about 96%, at least about 97%, at least about 98%, at least about 99%, or about 100% of cells in the isolated mammalian nucleus pulposus cell population express CD9 and CD109.

In some aspects, the isolated mammalian nucleus pulposus cells have an increased expression of CD9, CD109, or both CD9 and CD109 compared to the expression of CD9, CD109, or both CD9 and CD109 in premature nucleus pulposus cells. In some aspects, the isolated mammalian nucleus pulposus cells have an increased expression of nucleus pulposus-specific ECM genes compared to the expression of the same genes in premature nucleus pulposus cells. In some aspects, the isolated mammalian nucleus pulposus cells express one or more genes selected from the group consisting of ACAN, COL2A1, and COL6A3. In some aspects, the isolated mammalian nucleus pulposus cells have an increased expression of ACAN, COL2A1, and COL6A3 compared to the expression of ACAN, COL2A1, and COL6A3 in premature nucleus pulposus cells. In some aspects, the isolated mammalian nucleus pulposus cells comprise or consist essentially of mature mammalian nucleus pulposus cells. In some aspects, the isolated mammalian nucleus pulposus cells comprise or consist essentially of mature mammalian nucleus pulposus cells and the mature mammalian nucleus pulposus cells have an increased expression of nucleus pulposus-specific ECM genes relative to the expression of same genes in premature nucleus pulposus cells.

The isolated mammalian nucleus pulposus cell population in the disclosed cellular compositions can have the highest expression of nucleus pulposus-specific ECM genes relative to the expression of the same genes in the other NP cell population(s). For example, the isolated mammalian nucleus pulposus cell population in the disclosed compositions can expresses one or more genes selected from the group consisting of ACAN, COL2A1, and COL6A3. In some aspects, the isolated mammalian nucleus pulposus cell population in the disclosed compositions can expresses the one or more nucleus pulposus-specific ECM genes ACAN, COL2A1, and COL6A3 at a level between about 1.5 fold and about 10 fold greater than the expression level of the same genes in the other population(s) of NP cells.

In some aspects, the isolated mammalian nucleus pulposus cell population in the disclosed compositions can expresses ACAN at a level between about 1.5 fold and about 10 fold greater than the expression level of ACAN in the other population(s) of NP cells. In some aspects, the isolated mammalian nucleus pulposus cell population in the disclosed compositions can expresses ACAN at a level between about 1.5 fold and about 10 fold, between about 1.5 fold and about 9 fold, between about 1.5 fold and about 8 fold, between about 1.5 fold and about 7 fold, between about 1.5 fold and about 6 fold, between about 1.5 fold and about 5 fold, between about 1.5 fold and about 4 fold, between about 1.5 fold and about 3 fold, between about 1.5 fold and about 2 fold greater than the expression level of ACAN in the other population(s) of NP cells. For example, the isolated mammalian nucleus pulposus cell population in the disclosed compositions can expresses ACAN at a level of about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold greater than the expression level of ACAN in the other population(s) of NP cells.

In some aspects, the isolated mammalian nucleus pulposus cell population in the In some aspects, compositions can expresses COL2A1 at a level between about 1.5 fold and about 10 fold greater than the expression level of COL2A1 in the other population(s) of NP cells. In some aspects, the isolated mammalian nucleus pulposus cell population in the disclosed compositions can expresses COL2A1 at a level between about 1.5 fold and about 10 fold, between about 1.5 fold and about 9 fold, between about 1.5 fold and about 8 fold, between about 1.5 fold and about 7 fold, between about 1.5 fold and about 6 fold, between about 1.5 fold and about 5 fold, between about 1.5 fold and about 4 fold, between about 1.5 fold and about 3 fold, between about 1.5 fold and about 2 fold greater than the expression level of COL2A1 in the other population(s) of NP cells. For example, the isolated mammalian nucleus pulposus cell population in the disclosed compositions can expresses COL2A1 at a level of about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold greater than the expression level of COL2A1 in the other population(s) of NP cells.

In some aspects, the isolated mammalian nucleus pulposus cell population in the disclosed compositions can expresses COL6A3 at a level between about 1.5 fold and about 10 fold greater than the expression level of COL6A3 in the other population(s) of NP cells. In some aspects, the isolated mammalian nucleus pulposus cell population in the disclosed compositions can expresses COL6A3 at a level between about 1.5 fold and about 10 fold, between about 1.5 fold and about 9 fold, between about 1.5 fold and about 8 fold, between about 1.5 fold and about 7 fold, between about 1.5 fold and about 6 fold, between about 1.5 fold and about 5 fold, between about 1.5 fold and about 4 fold, between about 1.5 fold and about 3 fold, between about 1.5 fold and about 2 fold greater than the expression level of COL6A3 in the other population(s) of NP cells. For example, the isolated mammalian nucleus pulposus cell population in the disclosed compositions can expresses COL6A3 at a level of about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold greater than the expression level of COL6A3 in the other population(s) of NP cells.

In some aspects, the isolated mammalian nucleus pulposus cell population in the disclosed compositions can expresses ACAN, COL2A1, and COL6A3 at a level between about 1.5 fold and about 10 fold greater than the expression level of ACAN, COL2A1, and COL6A3 in the other population(s) of NP cells. In some aspects, the isolated mammalian nucleus pulposus cell population in the disclosed compositions can expresses ACAN, COL2A1, and COL6A3 at a level between about 1.5 fold and about 10 fold, between about 1.5 fold and about 9 fold, between about 1.5 fold and about 8 fold, between about 1.5 fold and about 7 fold, between about 1.5 fold and about 6 fold, between about 1.5 fold and about 5 fold, between about 1.5 fold and about 4 fold, between about 1.5 fold and about 3 fold, between about 1.5 fold and about 2 fold greater than the expression level of ACAN, COL2A1, and COL6A3 in the other population(s) of NP cells. For example, the isolated mammalian nucleus pulposus cell population in the disclosed compositions can expresses ACAN, COL2A1, and COL6A3 at a level of about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold greater than the expression level of ACAN, COL2A1, and COL6A3 in the other population(s) of NP cells.

In some aspects, the isolated mammalian nucleus pulposus cell population in the disclosed compositions has substantially no expression of any one of CD24, CD24, CD44, and Nanog. In some aspects, the isolated mammalian nucleus pulposus cell population in the disclosed compositions has no substantially increased expression level of any one of CD24, CD24, CD44, and Nanog relative to the expression level of these genes in the other population(s) of NP cells.

In some aspects, the protectant in the cellular composition can comprise a hydrogel. Hydrogels may be composed of one or more of the following constituents: hyaluronic acid, chitosan, collagen, gelatin, teleostean, alginate, cellulose, dextran, agarose, polyethylene glycol, polycaprolactone, acrylamide, fibrin, silk, polyeurathane, polylactic co-glycolic acid, chondroitin sulfate, and poly dimethylaminoethylmethacrylate.

In some aspects, the hydrogels may be self-assembling or require the addition of a cross-linking agent prior to delivery. The components may have undergone one or more chemical modifications.

In some aspects, the protectant can be a carrier. Suitable carriers include, e.g., saline, growth media, sera, buffers, and combinations thereof.

In some aspects, the protectant can be present in the composition at an amount between about 1% and about 50% volume of the protectant to volume of the composition (v/v). For example, the protectant can be present in the composition at an amount between about 1% and about 50%, between about 1% and about 45%, between about 1% and about 40%, between about 1% and about 35%, between about 1% and about 30%, between about 1% and about 25%, between about 1% and about 20%, between about 1% and about 15%, between about 1% and about 10%, or between about 1% and about 5% v/v of the composition. In some aspects, the protectant can be present in the composition at an amount of about 1%, about 2%, about 3%, about 5%, about 10%, about 15%, about 20%, about 25%, about 30%, about 35%, about 40%, about 45%, or about 50% v/v of the composition.

Also provided are a cellular compositions comprising a mammalian nucleus pulposus cell population having an ECM-generating phenotype and (i) expressing CD9 and having an anti-CD9 antibody bound to at least a portion of the cells in the population, or (ii) expressing CD109 and having an anti-CD109 antibody bound to at least a portion of the cells in the population. In some aspects, the cellular composition can have at least about 0.5%, about 1%, about 2%, about 3%, about 4%, about 5%, about 6%, about 7%, about 8%, about 9%, about 10% or more percent of cells in the composition labeled with an anti-CD9 antibody. In some aspects, the cellular composition can have at least about 0.5%, about 1%, about 2%, about 3%, about 4%, about 5%, about 6%, about 7%, about 8%, about 9%, about 10% or more percent of cells in the composition labeled with an anti-CD109 antibody. In some aspects, the cellular composition can have at least about 0.5%, about 1%, about 2%, about 3%, about 4%, about 5%, about 6%, about 7%, about 8%, about 9%, about 10% or more percent of cells in the composition labeled with an anti-CD9 antibody and an anti-CD109 antibody.

In some aspects, the population of isolated mammalian nucleus pulposus cells have an increased expression of CD9, CD109, or both CD9 and CD109 compared to the expression of CD9, CD109, or both CD9 and CD109 in premature nucleus pulposus cells. In some aspects, the population of isolated mammalian nucleus pulposus cells have an increased expression of nucleus pulposus-specific ECM genes compared to the expression of same genes in premature nucleus pulposus cells. In some aspects, the population of isolated mammalian nucleus pulposus cells have an increased expression of ACAN, COL2A1, and COL6A3 compared to the expression of ACAN, COL2A1, and COL6A3 in premature nucleus pulposus cells. In some aspects, the population of isolated mammalian nucleus pulposus cells have an increased expression of nucleus pulposus-specific ECM genes compared to the expression of same genes in premature nucleus pulposus cells.

Suitable anti-CD9 or anti-CD109 antibodies are known in the art and include readily available antibodies. For example, the anti-CD9 antibodies can be any one or more commercially available antibodies from Abcam® (Abcam plc, Cambridge, U.K.) having Catalog No. ab236630, ab263019, ab254175, ab2215, ab263023, ab223052, ab263024, ab58989, ab267502, ab267503, ab18241, ab187776, ab82394, ab239597, or ab82392; from Millipore Sigma® (MERCK KGAA, Darmstadt, Germany), having Catalog No. AB16969, SAB4700094, or MABT914; or from Thermo Fisher Scientific® (Thermo Fisher Scientific Inc., Waltham, MA), having Catalog No. 928-MSM1-P1, 928-MSM3-P1, 928-MSM3-P1ABX, or 928-MSM1-P1ABX. The anti-CD9 antibodies can be any one or more commercially available antibodies from Abcam®, having Catalog No. ab47169, ab235896, ab275953, or ab203588. In some aspects, the one or more anti-CD9 or anti-CD109 antibodies are labeled antibodies, such as antibodies that are labeled with a fluorophore, or attached to a bead.

Also disclosed herein are cellular compositions, comprising: isolated mammalian nucleus pulposus cells, wherein the isolated mammalian nucleus of pulposus cells of nucleus pulposus cells have an extracellular matrix (ECM)-generating phenotype, and a protectant. In some aspects, at least 90% of cells in the isolated mammalian nucleus pulposus cells express CD9. In some aspects, at least 90% of cells in the isolated mammalian nucleus pulposus cells express CD109. In some aspects, the isolated mammalian nucleus pulposus cells have the highest expression of CD9, CD109, or both CD9 and CD109 relative to the expression of CD9, CD109, or both CD9 and CD109 in other population(s). In some aspects, the isolated mammalian nucleus pulposus cells have the highest expression of nucleus pulposus-specific ECM genes relative to the expression of same genes in the other population(s). In some aspects, the isolated mammalian nucleus pulposus cells cam express one or more genes selected from the group consisting of ACAN, COL2A1, and COL6A3. In some aspects, the isolated mammalian nucleus pulposus cells can have the highest expression of ACAN, COL2A1, and COL6A3 relative to the expression of ACAN, COL2A1, and COL6A3 in other population(s). In some aspects, the isolated mammalian nucleus pulposus cells can comprise mature mammalian nucleus pulposus cells. In some aspects, the mature mammalian nucleus pulposus cells can have the highest expression of nucleus pulposus-specific ECM genes relative to the expression of same genes in the other population(s). In some aspects, the protectant can comprise a hydrogel. In some aspects, the protectant can be at least 50% volume to volume of the composition. In some aspects, the isolated mammalian nucleus pulposus cells can be derived from notochord-derived nucleus pulposus cells.

Further disclosed herein are cellular compositions comprising a population of mammalian nucleus pulposus cells having an extracellular matrix (ECM)-generating phenotype and (i) expressing CD9 and having an anti-CD9 antibody bound to at least a portion of the cells in the population, and/or (ii) expressing CD109 and having an anti-CD109 antibody bound to at least a portion of the cells in the population, and a protectant. In some aspects, the population of isolated mammalian nucleus pulposus cells can have the highest expression of CD9, CD109, or both CD9 and CD109 relative to the expression of CD9, CD109, or both CD9 and CD109 in other population(s). In some aspects, the population of isolated mammalian nucleus pulposus cells can have the highest expression of nucleus pulposus-specific ECM genes relative to the expression of same genes in the other population(s). In some aspects, the population of isolated mammalian nucleus pulposus cells can express one or more genes selected from the group consisting of ACAN, COL2A1, and COL6A3. In some aspects, the population of isolated mammalian nucleus pulposus cells can have the highest expression of ACAN, COL2A1, and COL6A3 relative to the expression of ACAN, COL2A1, and COL6A3 in other population(s). In some aspects, wherein the population of isolated mammalian nucleus pulposus cells can comprise mature mammalian nucleus pulposus cells. In some aspects, the mature mammalian nucleus pulposus cells can have the highest expression of nucleus pulposus-specific ECM genes relative to the expression of same genes in the other population(s). In some aspects, the cellular composition further comprise mesenchymal stem cells and/or aggrecan.

### Methods of Making the Compositions

Disclosed herein are methods of making the disclosed cellular compositions disclosed herein. The methods disclosed herein can include combining an isolated mammalian nucleus pulposus cell population having an ECM-generating phenotype with a protectant. Disclosed are method of makings a cellular composition, the method comprising: combining one or more isolated mammalian nucleus pulposus cells with a protectant, wherein the one or more isolated mammalian nucleus pulposus cells have an extracellular matrix (ECM)-generating phenotype.

In some aspects, the isolated mammalian nucleus pulposus cell population can be generated by isolating this cell population from other population(s) of nucleus pulposus cells.

In some aspects, the step of isolating can include methods selected from the group consisting of fluorescence-activated cell sorting, magnetic separation using negative selection with labeled beads, magnetic separation using positive selection with labeled beads, and other suitable cell isolation methods known in the art. In some aspects, the step of isolating can comprise labeling NP cells with anti-CD9 antibody and isolating nucleus pulposus cells that express CD9. In some aspects, the step of isolating can comprise labeling NP cells with anti-CD109 antibody and isolating nucleus pulposus cells that express CD109. In some aspects, the step of isolating can comprise labeling NP cells with anti-CD9 and anti-CD109 antibodies and isolating nucleus pulposus cells that express CD9 and CD109. In some aspects, the methods can comprise combining the isolated nucleus pulposus cells expressing CD9 and/or CD109 with a protectant.

In some aspects, the methods can comprise culturing the isolated nucleus pulposus cells expressing CD9 and/or CD109 in vitro.

In some aspects, the methods can comprise isolating and expanding in vitro the mammalian nucleus pulposus cell population having an ECM-generating phenotype. In some aspects, the cells are expanded in vitro by culturing the isolated cells in a medium comprising one or more growth factors selected from the group consisting of transforming growth factor (TGF)B1, TGFB3, fibroblast growth factor (FGF)1, FGF2, insulin growth factor (IGF)1, and epidermal growth factor (EGF). In some aspects, the methods comprise culturing the isolated mammalian nucleus pulposus cells in vitro under adherent and/or non-adherent conditions.

In some aspects, the methods can comprise obtaining a cellular composition comprising the isolated mammalian nucleus pulposus cell population that (i) expresses CD9 and has an anti-CD9 antibody bound to at least a portion of the cells in the population, or (ii) expresses CD109 and has an anti-CD109 antibody bound to at least a portion of the cells in the population.

Disclosed herein are methods of making cellular compositions. In some aspects, the methods can comprise: combining one or more isolated mammalian nucleus pulposus cells with a protectant, wherein the one or more isolated mammalian nucleus pulposus cells have an extracellular matrix (ECM)-generating phenotype. In some aspects, the one or more isolated mammalian nucleus pulposus cells can expand in vitro. In some aspects the isolated mammalian nucleus pulposus cells expanded in vitro can comprise an isolated mammalian nucleus pulposus cell population cultured in a medium comprising one or more growth factors selected from the group consisting of transforming growth factor (TGF)B1, TGFB3, fibroblast growth factor (FGF)1, FGF2, insulin growth factor (IGF)1 and epidermal growth factor (EGF). In some aspects, the methods can further comprise culturing mammalian nucleus pulposus cells in vitro under adherent and/or non-adherent conditions. In some aspects, the methods can further comprise isolating nucleus pulposus cells expressing CD9 and/or CD109. In some aspects, the methods can comprise culturing isolated nucleus pulposus cells expressing CD9 and/or CD109 in vitro. In some aspects, the method can further comprise combining isolated nucleus pulposus cells expressing CD9 and/or CD109 with a protectant. In some aspects, the isolated mammalian nucleus pulposus cell population (i) expresses CD9 and has an anti-CD9 antibody bound to at least a portion of the cells in the population, or (ii) expresses CD109 and has an anti-CD109 antibody bound to at least a portion of the cells in the population.

### Methods of Using the Compositions

Also disclosed herein are methods of treating a subject. In some aspects, the methods can comprise administering any of the disclosed compositions to the subject. Disclosed are methods of treating a subject, the method comprising administering one or more of the disclosed cellular compositions to the subject. In some aspects, the subject can be a mammal. In some aspects, the subject can be a human. In some aspects, the subject can have back pain. In some aspects, the disclosed methods can include administering the disclosed compositions to an intervertebral disc of the subject. In some aspects, the disclosed methods of using the compositions can comprise administering compositions comprising isolated allogeneic nucleus pulposus cells. In some aspects, the disclosed methods of using the compositions can comprise administering compositions comprising isolated autologous nucleus pulposus cells.

In some aspects, the administering of the disclosed compositions can include invasive or non-invasive methods administration. Examples of invasive methods of administration can include administering the compositions during or as part of a spinal surgery. Examples of non-invasive methods of administration can include administering the compositions with a percutaneous administration.

In some aspects, the volume of the disclosed compositions for administering to a subject can be, e.g., between about 10 μl and about 3000 μl, which values should be understood as being non-limiting. Suitable volumes can include between about 10 μl and about 3000 μl, about 100 μl and about 3000 μl, about 1000 μl and about 3000 μl, about 10 μl and about 2500 μl, about 100 μl and about 2500 μl, or about 1000 μl and about 2500 μl, such as about 100 μl, about 1000 μl, about 1500 μl, about 2000 μl, about 2500 μl, or about 3000 μl.

In some aspects, the number of cells in the disclosed compositions for administering to a subject can be, e.g., between about 10³ cells/ml and about 5×10⁶ cells/ml, which values should be understood as being non-limiting. Suitable numbers of cells in the compositions include between about 10³ cells/ml and about 5×10⁶ cells/ml, about 10³ cells/ml and about 5×10⁶ cells/ml, about 10³ cells/ml and about 5×10⁶ cells/ml, about 10⁴ cells/ml and about 5×10⁶ cells/ml, about 10⁵ cells/ml and about 5×10⁶ cells/ml, about 10⁶ cells/ml and about 5×10⁶ cells/ml, about 10³ cells/ml and about 4×10⁶ cells/ml, about 10³ cells/ml and about 3×10⁶ cells/ml, about 10³ cells/ml and about 2×10⁶ cells/ml, or about 10³ cells/ml and about 10⁶ cells/ml. Suitable numbers of cells in the compositions include about 10³ cells/ml, about 10⁴ cells/ml, about 10⁵ cells/ml, about 10⁶ cells/ml, about 2×10⁶ cells/ml, about 3×10⁶ cells/ml, about 4×10⁶ cells/ml, or about 5×10⁶ cells/ml.

Disclosed herein are methods of treating a subject, the method comprising administering to the subject a cellular composition comprising: isolated mammalian nucleus pulposus cells, wherein the isolated mammalian nucleus of pulposus cells of nucleus pulposus cells have an extracellular matrix (ECM)-generating phenotype, and a protectant. In some aspects, the subject can be a mammal. In some aspects, subject can be a human. In some aspects, the composition can further include mesenchymal stem cells and/or aggrecan. In some aspects, the subject can have back pain. In some aspects, the method can comprise administering the composition to an intervertebral disc of the subject. In some aspects, the composition can comprise isolated allogeneic nucleus pulposus cells. In some aspects, the composition can comprise isolated autologous nucleus pulposus cells.

Disclosed herein are methods of treating a subject, the method comprising administering to the subject a cellular composition comprising: a protectant, and isolated mammalian nucleus pulposus cells, wherein the isolated mammalian nucleus of pulposus cells of nucleus pulposus cells have an extracellular matrix (ECM)-generating phenotype. In some aspects, the cellular composition further comprise mesenchymal stem cells and/or aggrecan.

Disclosed herein are methods of treating a subject, the method comprising administering to the subject a cellular composition comprising: a population of mammalian nucleus pulposus cells and a protectant, wherein the population of mammalian nucleus pulposus cells have an extracellular matrix (ECM)-generating phenotype and (i) express CD9 and having an anti-CD9 antibody bound to at least a portion of the cells in the population, and/or (ii) express CD109 and having an anti-CD109 antibody bound to at least a portion of the cells in the population. In some aspects, the cellular composition further comprise mesenchymal stem cells and/or aggrecan.

In some aspects, the subject can be a mammal. In some aspects, subject can be a human. In some aspects, the subject can have back pain. In some aspects, the method can comprise administering the composition to an intervertebral disc of the subject. In some aspects, the composition can comprise isolated allogeneic nucleus pulposus cells. In some aspects, the composition can comprise isolated autologous nucleus pulposus cells.

### EXAMPLES

### Example 1. Single-Cell Transcriptomics Reveals Emergent Nucleus Pulposus Cell Subpopulations in the Postnatal Mouse Disc

### Materials and Methods

Single Cell RNA-Sequencing: To obtain notochord-derived NP cells for single cell RNA sequencing (scRNA-Seq), an Shh-cre;R26R-tdTomato mouse model was used, which leverages the fact that all cells of the embryonic notochord express Shh, and produces constitutive expression of tdTomato at the ROSA26 locus in these cells and their progeny (i.e. a fate map), including when Shh is no longer expressed. Mice were euthanized at 30 days-of-age, and disc cells isolated via mechanical dissociation and brief collagenase digestion. TdTomato+ cells were then purified using FACS. Libraries were generated using the Chromium controller (10X Genomics) and sequencing was performed using the Illumina HiSeq platform. Three replicate sequencing experiments were performed. Unsupervised clustering was conducted using Seurat, and differentiation trajectory analysis was performed using Monocle.

Histology: Lumbar spines were isolated from mice aged 0, 7, 14, 30 and 60 days, fixed in formalin, decalcified and processed for paraffin histology. Mid-sagittal sections were stained with Alcian blue and picrosirius red (ABPR) to demonstrate glycosaminoglycan (GAG) and collagen, respectively. Additionally, mid-sagittal, calcified cryosections were obtained from the lumbar spines of both Shh-cre;R26R:tdTomato and wild type mice at these same ages for fluorescent localization of NP cells and immunofluorescent localization of subpopulation-specific cell surface markers, respectively.

### Results

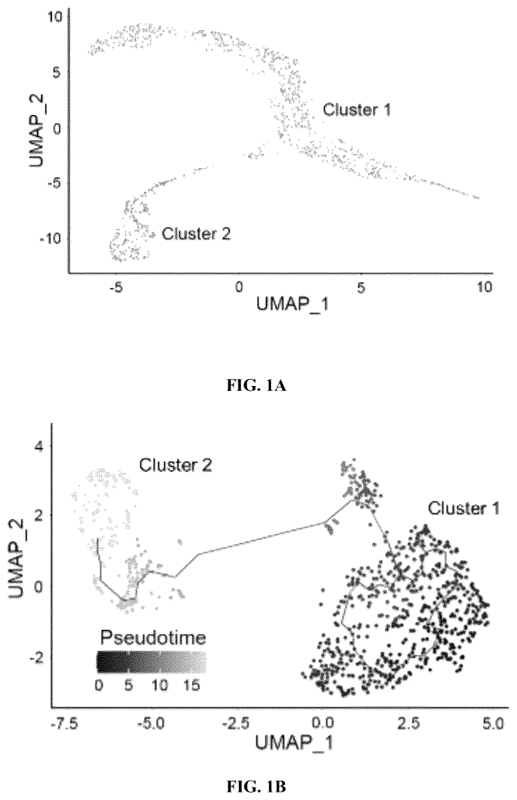

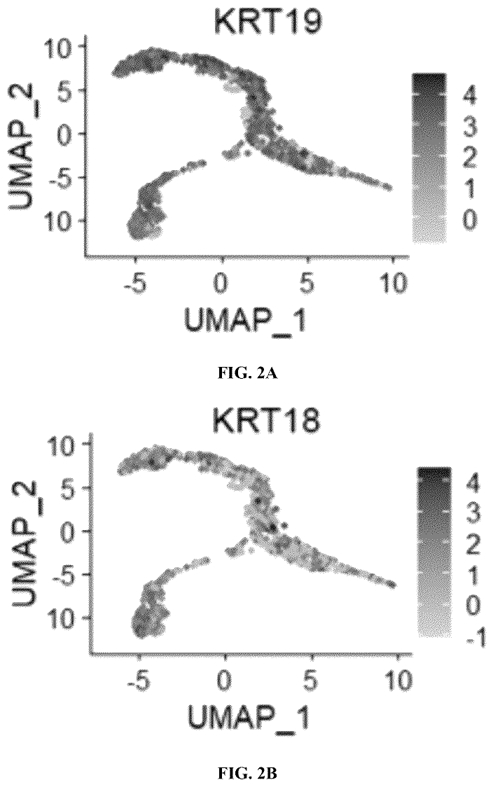

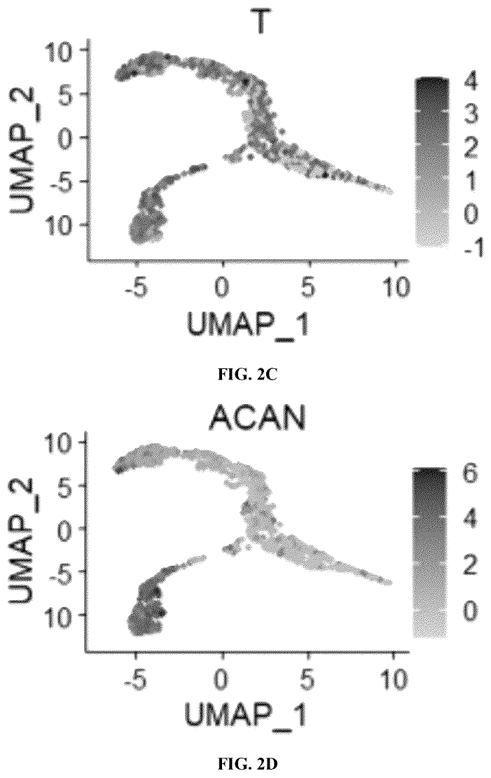

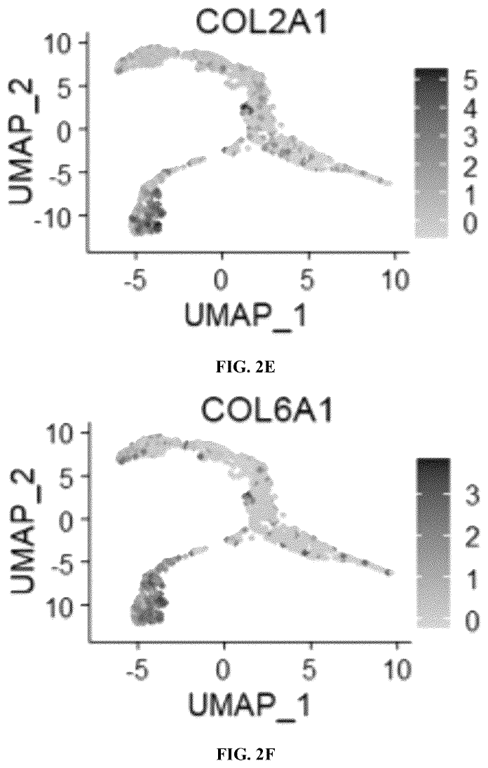

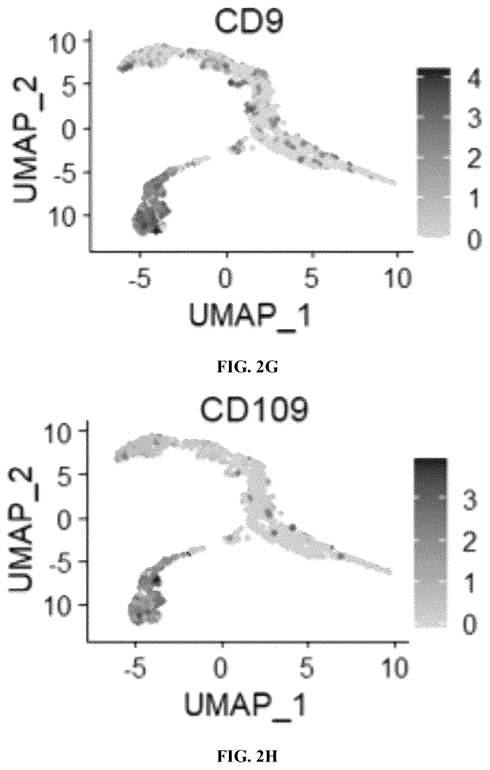

Single Cell RNA-Sequencing: The three replicate scRNA-Seq experiments exhibited high reproducibility, and results were pooled prior to further analyses. A total of 1116 notochord-derived NP cells (median 1445 genes/cell and 4854 UMIs/cell) were identified within the total sequenced cell population. UMAP plots revealed the presence of two distinct NP cell clusters exhibiting distinct gene expression profiles (FIG. 1A). Differentiation trajectory analysis showed that cells in clusters 1 and 2 aligned along a pseudo-timeline (FIG. 1B). Cluster-specific gene expression analyses demonstrated that NP cells in both clusters exhibited high expression of established NP markers including KRT18 and 19, and T, while expression of NP-specific ECM genes ACAN, COL2A1 and COL6A1 was confined to cells in cluster 2 (FIGS. 2A-2H) and Table 1.

TABLE 1
Expression of NP-specific ECM genes ACAN, COL2A1 and COL6A1
is statistically significantly greater in cluster 2 when
compared to those in cluster 1 of mouse NP cells.

| Cluster 2 FC vs: | Cluster 1 |
|---|---|
| ACAN | 14.64 |
| COL2A1 | 7.01 |
| COL6A1 | 4.23 |

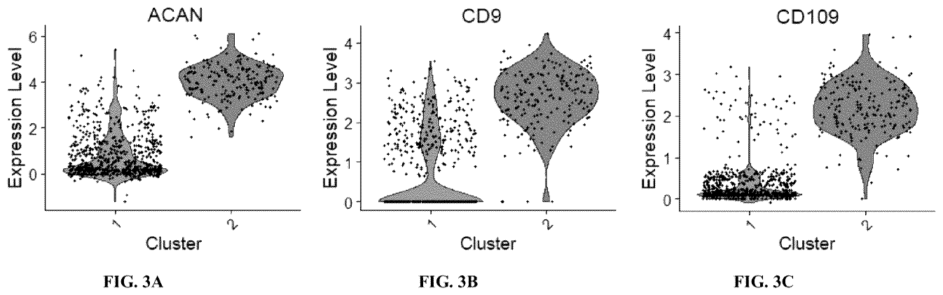

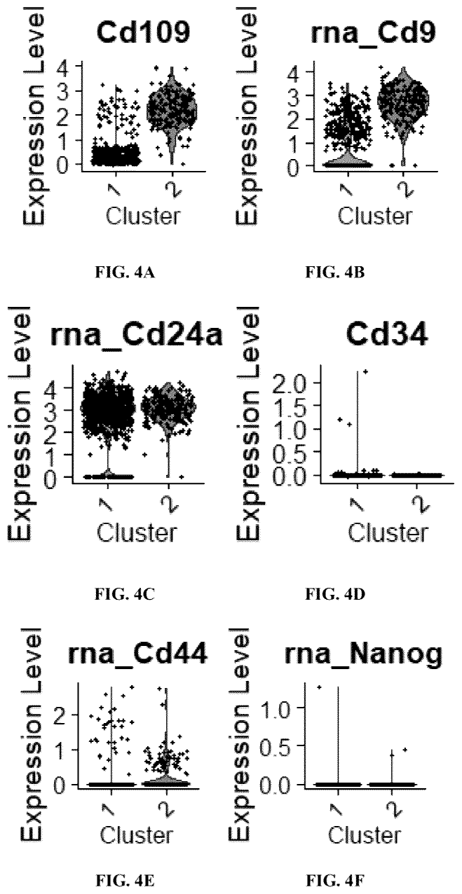

Based on the differential expression of these ECM genes, cells in clusters 1 and 2 were denoted early and late-stage NP cells, respectively. In addition to high ECM gene expression, late-stage NP-cells uniquely expressed the cell surface markers CD9 and CD109 (FIGS. 3A-3C and 4A and 4B). The scRNA-Seq dataset also showed no difference in CD24a expression between clusters 1 and 2, and absence of, or very low expression of, CD34, CD44, and Nanog genes in cluster 2 (FIGS. 4C-4F).

Cluster-specific gene expression analysis confirmed mutual high expression of CD-9, CD-109, and aggrecan by late-stage NP cells (FIGS. 3A-C).

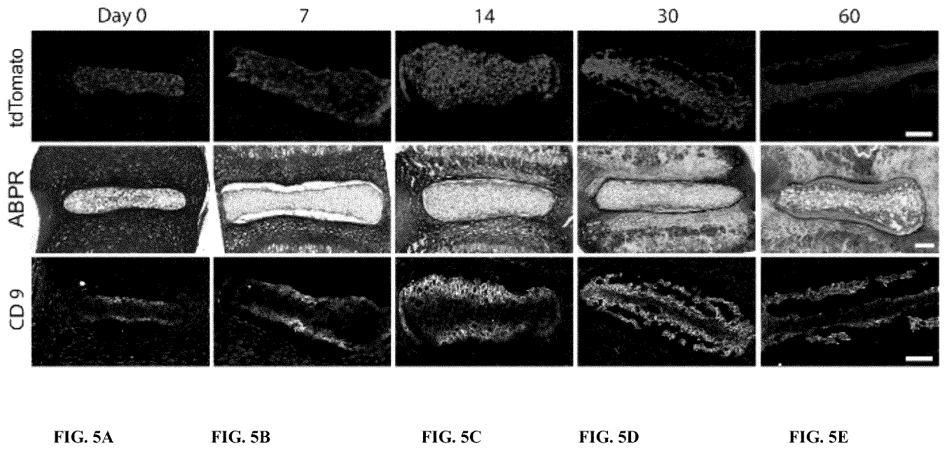

Histology: Fluorescence imaging revealed tdTomato expression throughout the NP. ABPR staining showed progressive ECM deposition in the NP from day 0 to 60. Notably, an emergent halo of GAG-rich ECM was apparent surrounding a central region of vacuolated cells. Immunofluorescent staining for CD9 demonstrated that late-stage NP cells co-localized with this emerging halo of GAG-rich ECM at all ages examined (FIG. 5A-5E).

In this study, the first evidence of emergent heterogeneity amongst NP cells in the postnatal mouse disc is provided. Specifically, the existence of early and late-stage NP cells was established, which exhibited distinct gene expression profiles reflecting different functional roles. The findings showed that early-stage NP cells residing at the center of the NP give rise to late-stage NP cells that reside at the periphery, and are responsible for producing the halo of proteoglycan-rich ECM. This ECM is important for maintaining disc hydrostatic pressure and resisting axial compressive forces. Importantly, the results also identified candidate surface markers, CD9 and CD109, which are uniquely expressed by late-stage NP cells. Histological findings showed that the transition from early to late-stage NP cells occurs progressively during postnatal growth. This may explain the eventual depletion of early stage (historically referred to as “notochordal” cells) in other species, including humans.

### Example 2. Effects of Organ Culture and Mesenchymal Stem Cell Delivery on the Cellular Composition of the Nucleus Pulposus

### Materials and Methods

Lumbar spines were obtained postmortem from three male large frame goats, and discs were isolated and allocated to 3 experimental conditions: 1) NP cells freshly isolated from discs postmortem (i.e., no organ culture); 2) NP cells isolated after 7 days of whole disc organ culture; and 3) NP cells and MSCs isolated after 7 days of whole disc organ culture. For this final group, allogeneic adult goat bone marrow-derived MSCs (0.2×10⁶ in 200 μl saline) were injected into the NPs of discs on day 0. For all conditions, cells were isolated from the NP using a central 5 mm biopsy punch followed by collagenase digestion. For organ culture, bony end plates were removed and discs were cultured with intact cartilaginous end plates in basal media (DMEM+10% FBS) under limited swelling conditions (Gawri, R., et al Spine (Phila Pa 1976) 36(22) (2011) pp 1835-42). For each condition, isolated cells from 5 discs were pooled, assessed for viability using trypan blue staining, and analyzed using scRNA-Seq. Libraries were generated using the Chromium controller (10X Genomics) and sequencing was performed using the Illumina HiSeq platform. Unsupervised clustering was conducted using Seurat and KEGG pathway analyses performed. Finally, one disc from each organ culture condition was assessed for cell viability using in situ live/dead staining.

### Results

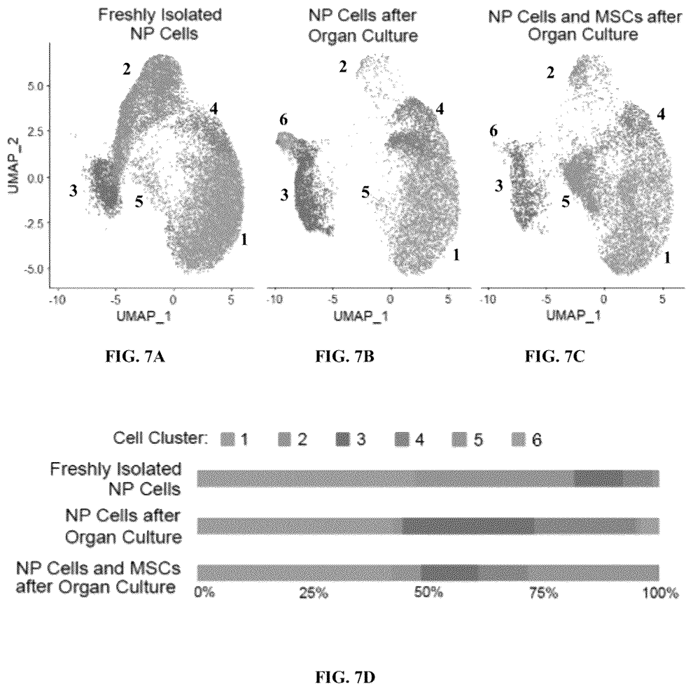

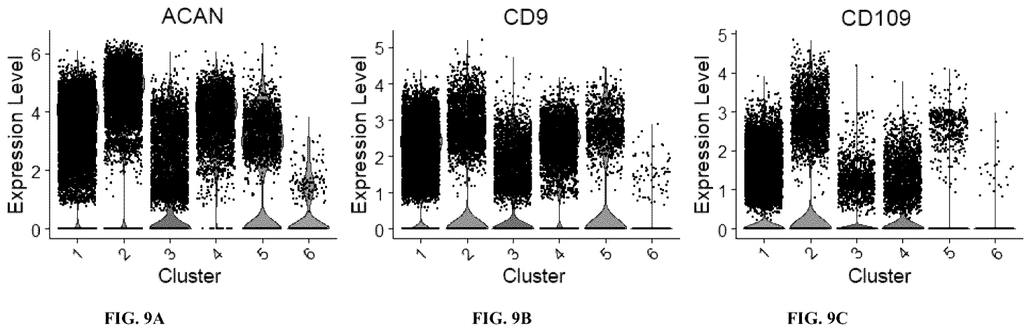

Goat Single Cell RNA-Sequencing: A total of 28,898 cells were sequenced (median 1284 genes/cell and 4904 UMIs/cell). UMAP plots identified 6 different cell clusters (FIGS. 7A-7C). Cluster-specific gene expression analyses performed for pooled cells revealed that expression of NP-specific extracellular matrix (ECM) genes, including aggrecan (ACAN), was highest in cluster 2 (FIG. 9A). Interestingly, similar to mouse findings, cells in cluster 2 concomitantly exhibited elevated expression of cell surface markers CD9 and CD109 (FIGS. 9B and 9C).

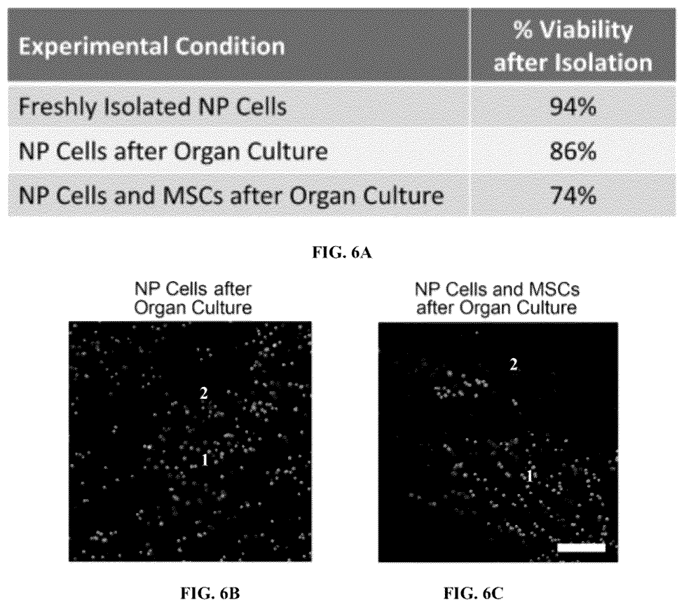

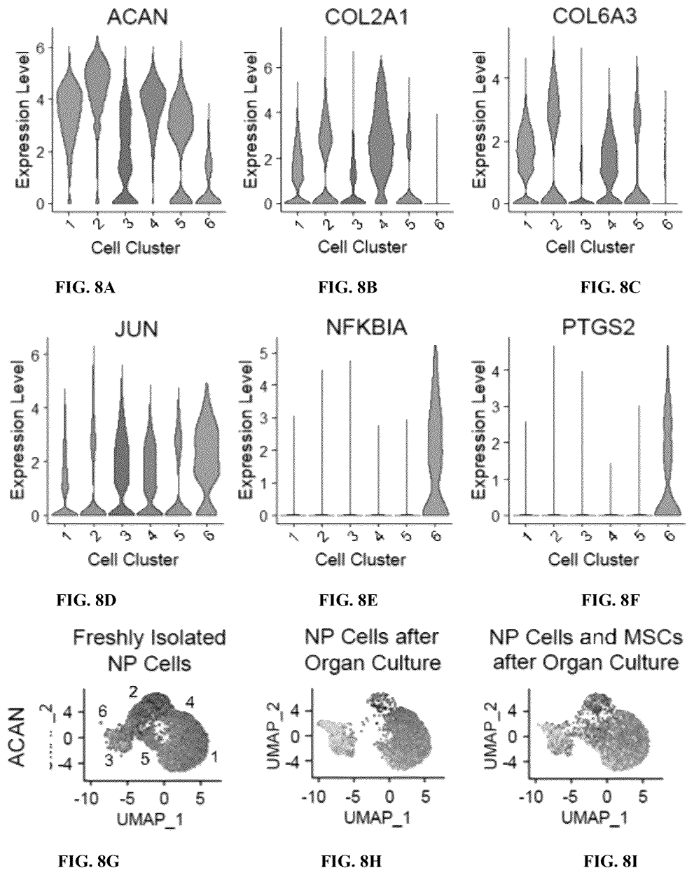

Cell viability and gene expression: A total of 28,898 cells were sequenced (median 1284 genes/cell and 4904 UMIs/cell): 13,697 freshly isolated NP cells (94% viability), 7,637 NP cells after organ culture (86% viability), and 7,566 NP cells and MSCs after organ culture (74% viability). Lower viability for both organ culture conditions was confirmed by in situ live/dead staining (FIGS. 6A-6C). UMAP plots generated for each condition using identical analysis parameters identified 6 different cell clusters (FIGS. 7A-7C), with the percentage of cells present within each cluster differing between conditions. The number of cells in cluster 1 remained relatively consistent between conditions. In contrast, the percentage of cells in cluster 2 in both organ culture conditions was greatly diminished (<7% of total cells). Cluster 5, largely absent in freshly isolated NP cells and NP cells alone after organ culture, comprised 28.15% of total cells for combined NP cells and MSCs after organ culture. In contrast, cluster 6, largely absent for freshly isolated NP cells, and combined NP cells and MSCs after organ culture, comprised 4.32% of total cells for NP cells alone after organ culture. Cluster-specific gene expression analyses performed for pooled cells across the 3 conditions (FIGS. 8A-8I) revealed that expression of NP-specific extracellular matrix (ECM) genes (ACAN, COL2A1 and COL6A3) was highest in cluster 2, and lowest in cluster 6 (Table 2).

TABLE 2
Expression of NP-specific ECM genes ACAN, COL2A1 and COL6A1
is statistically significantly greater in cluster 2 when
compared to those in other clusters of goat NP cells.

| Cluster | | | | | |
|---|---|---|---|---|---|
| 2 FC vs: | Cluster 1 | Cluster 3 | Cluster 4 | Cluster 5 | Cluster 6 |
| ACAN | 2.76 | 7.48 | 2.16 | 4.80 | 43.23 |
| COL2A1 | 3.39 | 4.38 | 0.56 | 3.40 | 8.72 |
| COL6A3 | 2.71 | 8.52 | 3.64 | 2.45 | 7.80 |

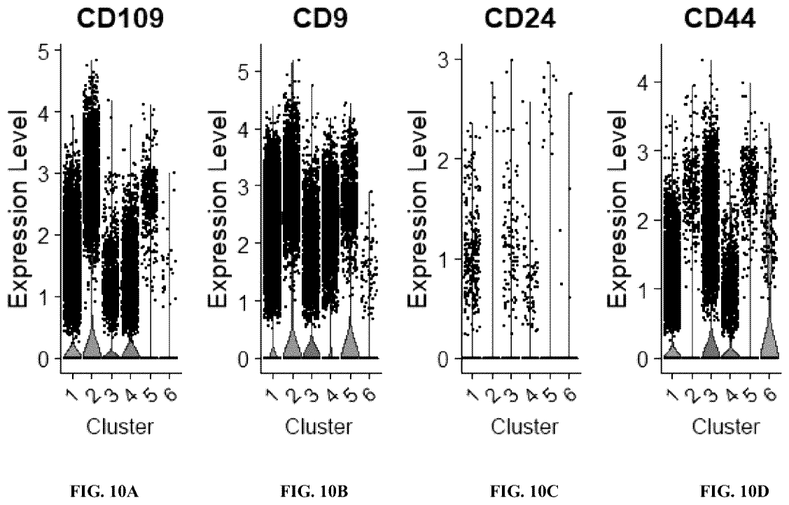

Expression of inflammatory/cell stress genes, including JUN, NFKBIA and PTGS2, was elevated in cluster 6 vs other clusters. Pathway analysis also revealed down regulation of ECM-related and PI3K-Akt signaling pathways in both clusters 5 and 6 compared to cluster 1, while there was upregulation of TNF-α signaling in cluster 6 vs cluster 5. The scRNA-Seq dataset also showed increased CD109 and CD9 expression in cluster 2 (FIGS. 10A and 10B), but no difference in CD24 gene expression between clusters, and no elevated expression of CD44 gene expression in cluster 2 (FIGS. 10C and 10D).

The goat is an established model for studying lumbar disc degeneration and cell-based therapies (Zhang, C., et al., Tissue Eng Part A 27(1-2) (2021) pp 117-128). This study provided evidence of cell heterogeneity with the NPs of adult goat intervertebral discs, including distinct subpopulations characterized by uncommon gene expression profiles, a result that is consistent with recent scRNA-Seq studies of both bovine and human discs showing similar heterogeneity (Fernandes, L. M., et al., Sci Rep 10(1) (2020) p. 15263; and Calio, M., et al. Int J Mol Sci 22(9) (2021)). Organ culture models are used extensively as preclinical tools to study disc degeneration and therapeutic intervention (Gawri, R., et al. Spine (Phila Pa 1976) 36(22) (2011) pp 1835-42; and Pfannkuche, J. J., et al. Connect Tissue Res 61(3-4) (2020) pp 304-321). This study showed that while organ culture did preserve some phenotypic properties of NP cells, there was an overall decrease in expression of important ECM genes after 7 days, due to cell stress and/or de-differentiation. While discs were maintained under limited swelling conditions in this study, using bioreactors that apply physiological loading during culture (Pfannkuche, J. J., et al. Connect Tissue Res 61(3-4) (2020) pp 304-321) may be able to better maintain native cell characteristics. Delivery of MSCs resulted in suppression of pro-inflammatory signaling, supporting a immunomodulatory role for these cells.

### Example 3. Co-Localization of CD-9 Positive NP Cells with Aggrecan and the Expression of CD-9 in Healthy and Degenerated Disc in Goats

Aggrecan is a proteoglycan that is an essential components of the healthy adult disc. The loss and/or degradation of aggrecan is one of the hallmarks of early disc degeneration.

### Materials and Methods

Immunofluorescent Imaging of Mouse Disc: Lumbar spines were isolated from Agc1tm(IRES-CreERT2); R26R-tdTomato mice aged 30, 60, 90 and 120 days, and mid-sagittal cryosections were stained for immunofluorescent co-localization of CD-9 with aggrecan expression.

Flow Cytometry of Human and Goat NP Cells: Healthy NP cells from adult human (commercially sourced) and goat donors were immunolabeled with an anti-CD9 antibody the percentage of CD-9 positive NP cells in each sample was analyzed by flow cytometry.

Immunohistochemistry of Healthy and Degenerate Goat Discs: With IACUC approval, moderate severity degeneration was induced in the lumbar discs of adult goats via injection of 1 U chondroitinase ABC (Peck, S. H., et al. Sci Rep 7(1) (2017) pp 10504). After 12 weeks, degenerate (n=4) and adjacent healthy control (n=3) discs were harvested postmortem, fixed, decalcified and processed into paraffin. Sections were immunostained for CD-9 and imaged using bright field microscopy. The percent of CD-9 positive NP cells was quantified, with significant differences determined using an unpaired t-test.

### Results

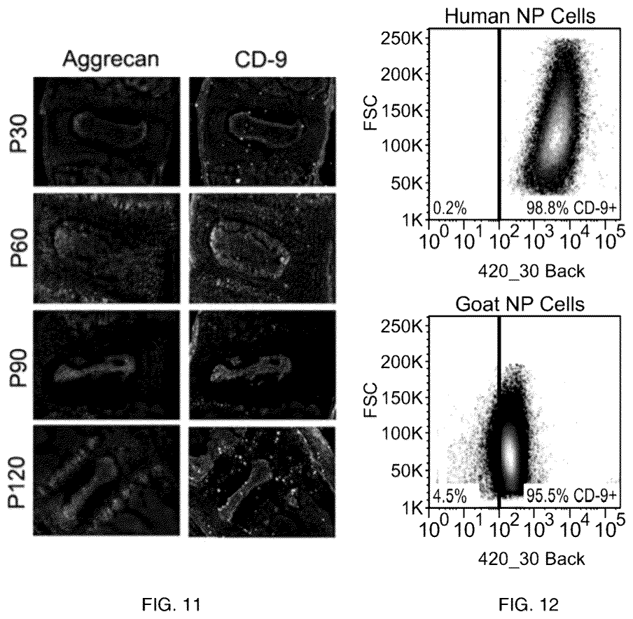

Fluorescence imaging revealed emergent tdTomato-positive aggrecan-expressing cells localized to the peripheral region of the postnatal mouse NP, and immunofluorescent staining demonstrated that these same cells co-expressed CD-9 (FIG. 11). No CD-9 expression was observed in the annulus fibrosus.

Flow cytometry analysis revealed that healthy adult human and goat NP cells were found to be 99.8 and 95.6% immunopositive for CD-9, respectively (FIG. 12).

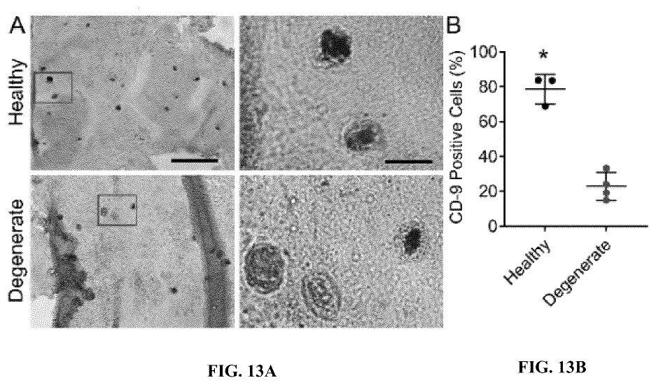

CD-9 positive NP cells were observed in the NPs of both healthy and degenerate discs; however the percentage of CD-9 positive cells was significantly lower in degenerate compared to healthy discs (23% vs 79%, respectively, p<0.05, FIG. 13).

### Example 4. Protocol for Isolation of CD-9 Positive NP Cells from NP Tissue and the Administration of the Cells to Patients

Waste NP tissue can be obtained from a subject(s) undergoing spinal procedures (autologous cells), or donor tissue, surgical or postmortem (allogeneic cells).

NP cells can then isolated via enzymatic digestion (e.g. via collagenase treatment), or tissue is placed in the culture disc, allowing cells to migrate out.

NP cells can then labeled with fluorescently tagged antibodies against CD9 and/or CD109.

CD9+, CD109+ or CD9+/CD109+ NP cells can be isolated using fluorescence activated cell sorting.

Isolated CD9+, CD109+ or CD9+/CD109+ NP cells can be expanded for 1 to 14 days in the absence or presence of growth factors, including TGF-b1, TGF-b3, FGF1, FGF2, IGF1, and EGF individually, sequentially or in combination, at concentrations ranging from 1-100 ng/ml of culture media. Alternatively, cells can be expanded in the presence of serum (0-30%).

Expanded cells can then be suspended in sterile saline, or vehicle such as hydrogel or fibrin glue, at concentrations ranging from 1000 to 10,000,000 cells per ml. Optionally, cells can be combined with mesenchymal stem cells (autologous or allogeneic) and/or aggrecan in the same delivery vehicle at concentrations ranging from 1000 to 10,000,000 cells per ml. The expression and purification of aggrecan is described for example in Seyfried et al., J Biol Chem, 2005, 280; 5435-48.

Cells (suspended in vehicle) can be injected into the disc nucleus pulposus of one or multiple discs of a subject. Delivery can be via percutaneous injection under minimal sedation, or as part of an invasive procedure under general anesthesia. Injection volumes can range from 10 ul to 1 ml per disc. Subjects in need of such treatment can include patients with intervertebral disc degeneration and/or concomitant low back pain.

Cell injections as described above can be performed once, or multiple times at intervals ranging from one month to 1 year.

### Example 6. Pigs Retain Notochordal and Mature Nucleus Pulposus Cells

Pigs represent a translational animal model for evaluating the therapeutic potential of CD9+ nucleus pulposus cells, as unlike most large animals (and humans), pigs retain significant numbers of both notochordal (progenitor) and mature cells (putatively CD9- and CD9+ cells, respectively) throughout growth and adulthood. This was confirmed experimentally as follows.

Juvenile (9 week old) porcine spines were sourced from the slaughterhouse and the exterior sterilized with 70% ethanol for 30 minutes. Subsequently, the nucleus pulposus (NP) tissues were dissected from the thoracic and lumbar intervertebral discs and processed for cell isolation. NP tissues were digested with 3 mg/ml collagenase type 1 (Worthington Biochemical Corporation, USA), for 3-hours, followed by centrifugation at 500 g for 10 minutes. The resulting cell pellet was resuspended in 0.25% Trypsin EDTA (Thermo Fisher Scientific, USA) and incubated for 10 minutes in a 37° C. water bath. After digestion, 1 ml of fetal bovine serum (Thermo Fisher Scientific, USA) was added to stop trypsin activity, and the cell suspension was filtered using a 100-micron cell strainer. The filtrate was centrifuged at 500 g for 10 minutes, and the resulting cell pellet was resuspended in PBS and used for flow cytometry. The cell suspension was divided into two labeled tubes: 1. Unlabeled and 2. CD9 labeled. A 10 μl of anti-CD9 antibody (Abcam, USA) was added to the CD9 labeled tube and incubated for 30-minutes at room temperature in the dark. Subsequently, 3 ml of PBS was added to the tube and centrifuged at 500 g for 10 minutes. Finally, the cell pellet was resuspended in 200 μl PBS and the percentage of CD9 positive cells in the samples was determined using a flow cytometer (BD LSR II; BD Biosciences, USA) and Flowjo software.

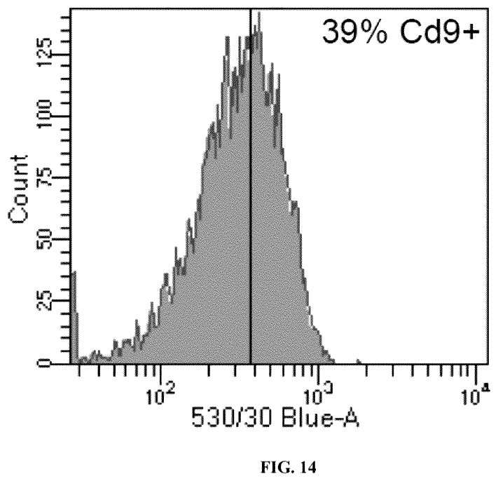

The results (FIG. 14) demonstrated that 39% of porcine NP cells expressed Cd9, indicating these cells to be mature, extracellular matrix producing cells.

### Example 7. Single Cell RNA Sequencing Reveals Emergent Notochord-Derived Cell Subpopulations in the Postnatal Nucleus Pulposus

### Materials and Methods

Animals and Study Design. To obtain notochord-derived NP cells for scRNA-Seq and for in situ validations experiments, the Shh-Cre:R26R-tdTomato (Shh/tdTomato) mouse model was used, which leverages the fact that the cells of the embryonic notochord express sonic hedgehog (Shh), and produces constitutive expression of tdTomato at the ROSA26 locus in these cells and their progeny, including when Shh is no longer expressed. Shh/tdTomato mice were generated by breeding Shh-Cre mice (RRID:IMSR_JAX:005622; Jackson Laboratory, Bar Harbor, ME, USA)25 with R26R-tdTomato mice (RRID:IMSR_JAX:007905; Jackson Laboratory). Animals were euthanized via carbon dioxide inhalation and spines were harvested for the experiments described. Single cell sequencing was performed for postnatal day (P)30 animals, while validation experiments were performed at ages spanning birth to P90.

To examine how the number of CD9+ NP cells changes as a function of disc degenerative condition, an established goat model of lumbar disc degeneration was used. Moderate severity degeneration was induced in the lumbar discs (L1-2 to L4-5) of six, 2-year-old large frame goats via injection of 1 U chondroitinase ABC (ChABC) into the NP (Gullbrand, S E, et al. Osteoarthritis and cartilage 2017; 25:146-156). Briefly, under general anesthesia, the lumbar spine was exposed using an open, lateral, retroperitoneal transpsoatic approach. ChABC was suspended in 200 μl of sterile saline and injected into the NP using a 22G spinal needle under fluoroscopic guidance. This needle size does not, on its own, induce detectable degeneration (Gullbrand, S E, et al. Osteoarthritis and cartilage 2017; 25:146-156). Adjacent, non-degenerate disc levels (T13-L1 and L5-6) were maintained as healthy, non-degenerate controls. The incision was closed in layers, and the animal recovered and returned to housing. For analgesia, animals were administered per-operative transdermal fentanyl (2.5 mcg/kg/hr) and intravenous flunixin meglumine (Banamine, 1.1 mg/kg), while florfenicol (40 mg/kg) was administered for antimicrobial prophylaxis. Animals were housed in groups with unlimited access to exercise, and evaluated daily by a veterinarian for signs of pain, behavior changes or gait abnormalities for the duration of the study. A total of 4 degenerate and 3 healthy control discs were used for this study, with remaining discs allocated to other, unrelated studies. Twelve weeks after inducing degeneration, animals were euthanized via an overdose of sodium pentobarbital and lumbar spines harvested for the experiments described below.

Cell Isolation, Single Cell RNA-Sequencing and Analysis. Spinal columns were excised from P30 mice postmortem. Under a dissecting microscope, dorsal boney elements and the spinal cord were removed leaving the isolated ventral spinal column comprising the vertebrae and discs. Whole discs in the cervical, thoracic and lumbar regions (˜15 discs) were carefully isolated by cutting through the vertebral boney endplates with a scalpel, and bisected to expose the NPs. Isolated discs were placed in a phosphate buffered saline containing 25 mM HEPES and 0.1% FBS, finely diced, then digested in collagenase (1 ml/mg; Sigma-Aldrich, St Louis, USA) for 30 mins. After digestion, cells were strained through a 50 μm filter. TdTomato+ cells were then enriched using fluorescence activated cell sorting 25 Cells from ˜10 litter-matched Shh-Cre:tdTomato mice were pooled for sequencing.

Single cell sequencing of enriched tdTomato+ cells was performed using methods similar to those described previously (Zhong, L, et al. Elife 2020; 9). The experiment was performed in triplicate. Viability (˜95%) was confirmed using trypan blue staining and three batches of single cell libraries were generated using the 10× Genomics Chromium Single Cell 3′ Reagent kit v3 per manufacturer's instructions. Libraries were uniquely indexed using the Chromium dual Index Kit, and paired-end sequencing was performed at a targeted depth for each library of 20,000 mean reads per cell (NovaSeq 6000; Illumina, San Diego, USA). Cellranger (Version 3.0.2, 10 Genomics) was used to demultiplex reads, followed by extraction of cell barcode and unique molecular identifiers (UMIs), and the cDNA insert was aligned to a modified reference mouse genome (mm10). Poor quality cells (<200 genes or >5% mitochondrial genes) and doublets (>6000 genes) were excluded. Gene expression was natural long transformed and normalized using the Seurat v4 toolkit and R Statistical Software (v4.1.2; R Core Team 2021) (Hao, Y, et al. Cell 2021; 184:3573-3587 e3529).

Results from the three sequencing batches were integrated for analysis. Unsupervised clustering was initially performed for the entire sequenced cell population and visualized using Uniform Manifold Approximation and Projection (UMAP) plots. Cell populations were identified using known markers for NP, mesenchymal, and hematopoietic, endothelial and blood cell markers. Unsupervised clustering was subsequently repeated for the cell subpopulation identified as NP cells and visualized using UMAP plots. Significant differences in gene expression between identified subpopulations of NP cells were determined. Cell cycle analysis was performed for NP cell subpopulations using cell cycle phase marker genes (Tirosh, I, et al. Science 2016; 352:189-196) in the CellCycleScoring package. Cells in the G1 phase were classified as resting, while those in the G2M or S phases were classified as proliferating. Differentiation trajectory analysis was performed for NP cell subpopulations using the Monocle 3 package (RRID:SCR_018685) (Trapnell, C, et al. Nat Biotechnol 2014; 32:381-386). The RNA velocity of NP cells in each cluster was calculated using the packages ‘velocity’ and ‘scVelo’ (RRID:SCR_018168) (Bergen, V, et al. Nat Biotechnol 2020; 38:1408-1414). To determine biological pathways enriched in specific NP cell subpopulations, gene ontology (GO) and Kyoto Encyclopedia of Genes and Genomes (KEGG) analyses were performed using the clusterProfiler (v4.5.3; RRID:SCR_016884) package for significantly differentially expression genes with a log₂ fold change ≥1. Protein-Protein Interaction (PPI) Network Construction and Module Analysis was used to obtain interaction relationships among differentially expressed genes (Szklarczyk, D, et al. Nucleic Acids Res 2021; 49: D605-D612), with the minimum required interaction score set to high confidence (0.7). Examination of cell-cell interactions within and between different NP cell subpopulations was performed using the CellChat tool (RRID:SCR_021946) (Jin, S, et al. Nat Commun 2021; 12:1088). Using the paracrine/autocrine signaling interaction dataset of CellChatDB as the referencing database, the communication probability was computed using a truncated mean of 20%. Cell-cell communication was subsequently inferred and the cell-cell communication network was aggregated with default parameters. The number of interactions was visualized to show the aggregated cell-cell communication network and signaling from each cell cluster.

Histology. Lumbar spines (vertebral columns with posterior boney elements removed) were isolated postmortem from Shh/tdTomato mice at P0, P7, P14, P30 and P60 (n=3 per age), fixed in buffered 10% formalin overnight and decalcified in formic and ethylenediaminetetraacetic acids for 24 hours (Formical 2000; StatLab Medical Products, Mckinney, USA). For fluorescent and immunofluorescent localization of NP cells, spines were then immersed into 20% sucrose and 2% polyvinylpyrrolidone (PVP) at 4° C. overnight, embedded in OCT compound (Sakura), and mid-sagittal 10 μm sections were cut on a freezing microtome (NX-70) Cryostar; Thermo Fisher Scientific Inc; Waltham, MA). For immunofluorescence, sections were incubated with primary antibodies against rabbit anti-Krt19 (1:200; Cat#NB100-687, RRID:AB_2265512; Novus Biologicals, Centennial. USA), rat anti-Cd9 (1:200; Cat#ab82390, RRID:AB_2244514; Abcam Inc) or rabbit anti-Cd109 (1:200; Cat#ab203588, RRID:AB_2936927; Abcam Inc) at 4° C. overnight, followed by Alexa Fluor 488-conjugated donkey anti-rabbit (1:1000; Cat#A-21206, RRID:AB_2535792; Thermo Fisher Scientific) or Alexa Fluor 647 goat anti-rat (1:1000, Cat#A-21247, RRID:AB_141778; Thermo Fisher Scientific) secondary antibodies for 1 hour at room temperature. For negative controls, primary antibodies were omitted. Finally, sections were counterstained with DAPI (Fluoromount-G; SouthernBiotech; Birmingham. USA) and imaged using a confocal microscope (LSM 9080; Carl Zeiss A G; Oberkochen, Germany). For histochemical assessment of progressive ECM deposition in mouse discs, lumbar spines (n=3 per age) were fixed and decalcified as above, and then processed into paraffin. Mid-sagittal, 10 μm sections were cut and double stained with Alcian blue and picrosirius red for qualitative assessment of glycosaminoglycan and collagen distribution, respectively, and imaged using bright field microscopy (Eclipse 90i; Nikon; Tokyo, Japan).

For goat samples, vertebra-disc-vertebra segments were fixed in 10% formalin, completely decalcified in formic and ethylenediaminetetraacetic acids, and processed into paraffin. For semi-quantitative grading of histological condition, mid-sagittal, 8 μm-thick sections were cut and double stained with Alcian blue (glycosaminoglycans) and picrosirius red (collagen). Semi-quantitative histological grading was performed by 3 blinded assessors (averaged) (Gullbrand, S E, et al. Osteoarthritis and cartilage 2017; 25:146-156). Parameters assessed included NP cellularity, NP ECM, endplate structure, NP-AF boundary, and AF organization, each on a continuous scale from 0 (best) to 100 (worst). An overall grade (ranging from 0 to 500) was calculated as the sum of these individual scores. For immunohistochemical assessment of CD9 expression on goat sections, 8 μm sections were rehydrated, and antigen retrieval performed at 60° C. in a Tris-EDTA pH 8.0 buffer, overnight. Endogenous peroxidase activity was blocked by treating sections with 3% hydrogen peroxide for 12 minutes, followed by Background Buster (Innovex Biosciences; Richmond, USA) for 10 minutes at room temperature to block non-specific protein binding. Sections were then incubated with rabbit anti-CD9 (1:200, Cat#ab223052, RRID:AB_2922392, Abcam Inc) at 4° C. overnight. Staining was visualized using the Vectastain Elite ABC-Peroxidase Kit (Vector Laboratories; Burlingame, USA) and diaminobenzidine chromogen (Thermofisher Scientific; Waltham, USA) according to the manufacturer's protocol. Finally, sections were counterstained with hematoxylin QS (Vector Laboratories; Burlingame, USA) and cover-slipped with aqueous mounting medium (Agilent; Santa Clara, USA). For analysis, the slides were imaged under bright field light microscopy. Three randomly selected regions of interest within the NP were analyzed. For each region, the number of immunopositive cells was counted and normalized as a percentage of the total number of cells present. Cell counting for each region was performed by three individuals who were blinded to the study groups and averaged across scorers prior to statistics.

Flow Cytometry. Notochord-derived NP cells were enriched from Shh/tdTomato mice (cells from ˜6 animals pooled per timepoint) at P0, P30, P60 and P90 as described. Cells were stained sequentially with a rabbit polyclonal Krt19 antibody conjugated to Alexa Fluor 647 (1:200; Cat#NB100-687AF647, RRID:AB_11016710; Novus Biologicals) and a rat monoclonal CD9 antibody conjugated to Alexa Fluor 750 (1:200; Cat#NBP1-44876AF750, RRID:AB_10008106; Novus) for 45 minutes on ice. Cells were then washed twice in phosphate buffered saline containing 2% fetal bovine serum (ThermoFisher Scientific; Waltham, MA) and analyzed using a flow cytometer (LSRII; BD Biosciences; Franklin Lakes, USA). NP cells were identified within the total population as double positive for tdTomato and Krt19, and CD9+ cells as a percentage of this NP cell population at each age were determined using Flowjo software (BD Biosciences). Unstained cells served as negative controls. Additionally, NP cells from healthy adult goat discs were analyzed. NP tissue was isolated from three lumbar discs of a healthy 2-year old adult goat, digested for 1 hour in 2.5 mg/ml pronase, followed by 4 hours in 0.5 mg/ml collagenase, both at 37 degrees, then filtered through a 70 micron cell strainer. Cells from the three discs were pooled, stained with a rat monoclonal CD9 antibody conjugated to Alexa Fluor 750 and analyzed using a flow cytometer. The number of CD9+ cells as a percentage of total NP cells was determined using Flowjo.

Statistical Analyses. Statistically-significant differences in histological grade of degeneration and the percentage of NP cells immunopositive for CD9 between healthy and degenerative goat discs were established using Student's t-tests using Prism software (GraphPad Software, San Diego, CA). Data is presented as mean ± standard deviation and significance was defined as p<0.05.

### Results

### Global Single Cell RNA Sequencing Findings

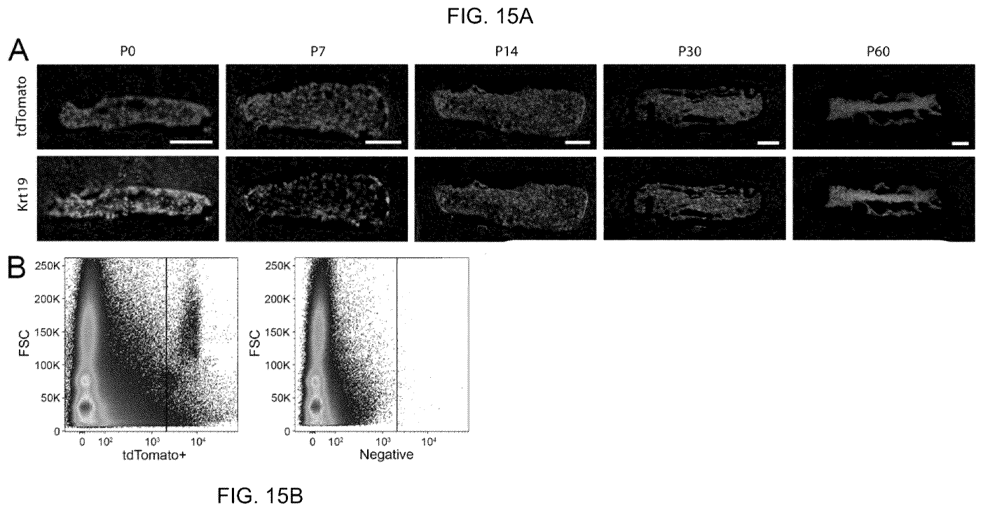

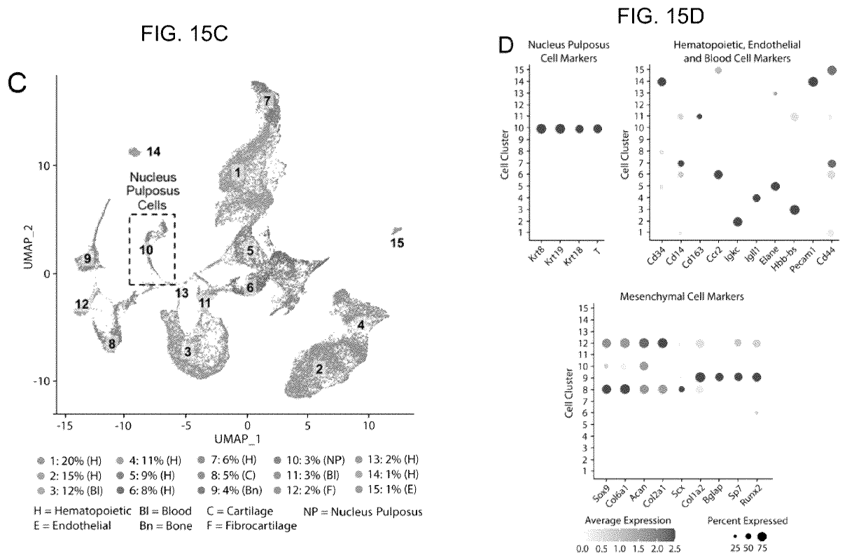

Nucleus pulposus-specific tdTomato expression in Shh/tdTomato mice was confirmed through in situ fluorescence imaging of lumbar discs from birth to postnatal day (P)60, which demonstrated that tdTomato was confined to the NP (FIG. 15A). Immunofluorescent staining on the same histological sections demonstrated that putative NP cells co-expressed the NP specific marker cytokeratin 19 (Krt19)34 at each age examined, confirming their identity. For single cell RNA sequencing, cells were isolated through gross dissection of discs from cervical, thoracic and lumbar spines pooled from litter-matched P30 Shh/tdTomato mice, followed by brief collagenase digestion and fluorescence-activated cell sorting (FIG. 15B). Three complete replicate scRNA-Seq experiments were performed and the results were pooled prior to analysis.

After quality control, a total of 27,066 cells with a median of 2311 genes/cell and a median of 11,660 unique molecular identifiers (UMIs)/cell were identified. Unsupervised clustering identified 15 unique cell subpopulations (FIG. 15C). Surprisingly, despite enriching for tdTomato+ cells, the sequenced cell population was heterogenous and included significant numbers of hematopoietic and mesenchymal lineage cells identified through cluster-specific expression of established markers (FIG. 15D). This unexpected heterogeneity may be in part due to leakage and transfer of tdTomato protein from notochordal cells to adjacent cell populations, which is detectable by the laser during cell sorting even when present at low levels. Nucleus pulposus cells, identified through cluster-specific expression of cytokeratins (Krt) 8, 18 and 19, and brachyury (T) comprised 3% of the total cell population (FIG. 15D).

### Identification of Nucleus Pulposus Cell Subpopulations

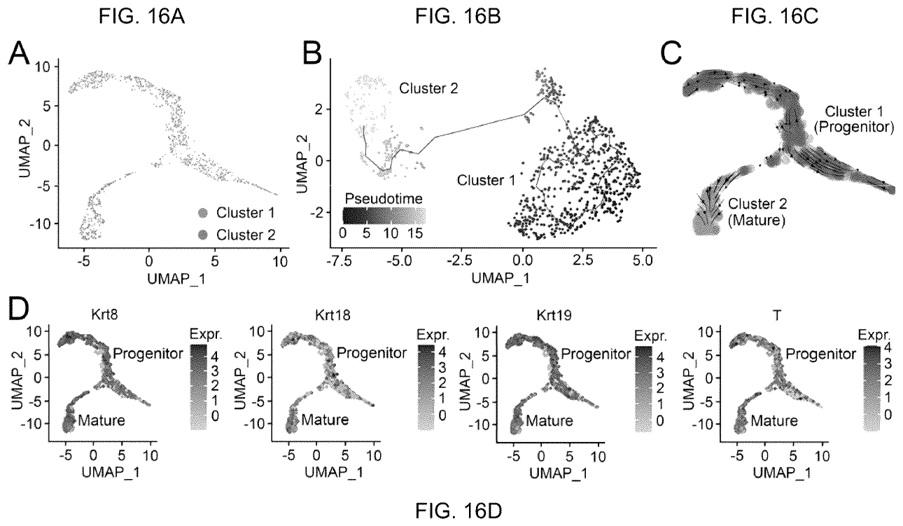

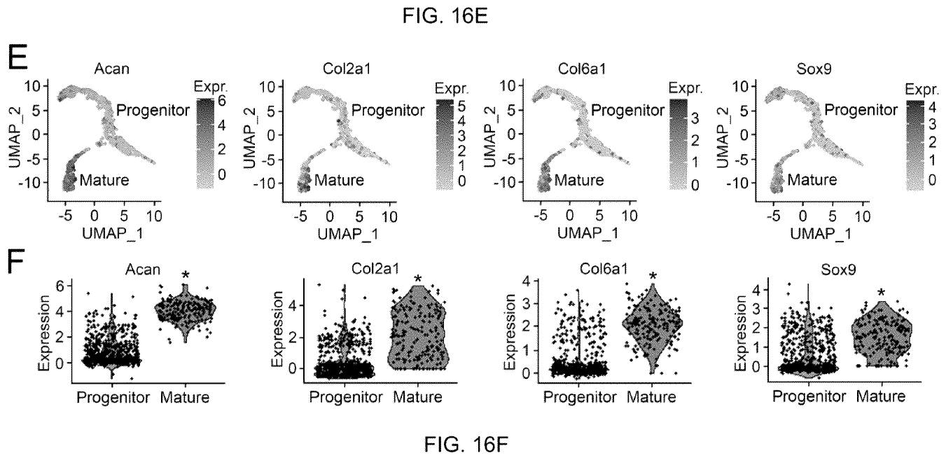

Using the above NP marker genes, a total of 936 notochord-derived NP cells with a median of 1445 genes/cell and a median of 4854 UMIs/cell were identified within the total sequenced cell population. Focusing solely on these NP cells, the unsupervised clustering was repeated, identifying two distinct NP cell subpopulations (clusters 1 and 2, comprising 76% and 24% of NP cells, respectively, FIG. 16A). Differentiation trajectory analysis was used to stratify these subpopulations, with cells in clusters 1 and 2 aligning along a pseudo-timeline (FIG. 16B). Additionally, RNA velocity analysis demonstrated that the direction of differentiation was predominantly towards cluster 2 (FIG. 16C). Based on this relationship, cells in clusters 1 and 2 were labeled “progenitor” and “mature” NP cells, respectively.

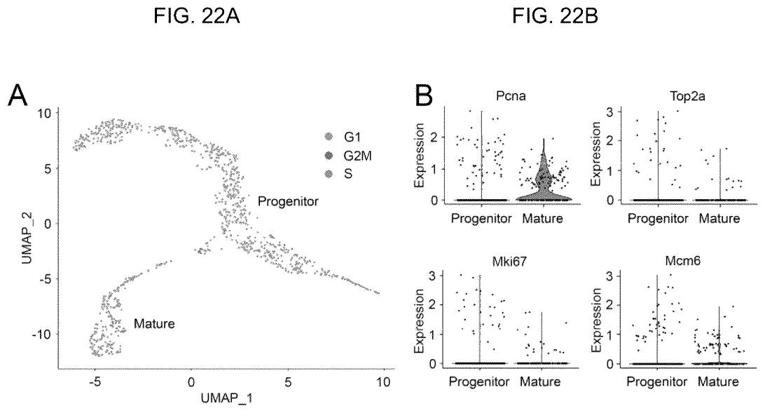

There were a total of 317 genes that exhibited significantly different expression levels in mature versus progenitor NP cells. Of these, 274 genes were upregulated (Table 3) and 43 genes were downregulated (Table 4). With respect to NP specific markers Krts 8, 18 and 19, and T, these were expressed at similar levels across both cell clusters (FIG. 16D), with the exception of Krt18, which was modestly but significantly (0.61 log2 fold) higher in mature NP cells. Amongst the top upregulated genes in mature NP cells were those encoding established components of the healthy NP ECM (FIGS. 16E and F), including aggrecan (Acan, 4.11 log2 fold higher), collagen II-alpha 1 (Col2a1, 3.25 log2 fold higher) and collagen VI-alpha 1 (Col6a1, 2.2 log2 fold higher), in addition to the master chondrogenic transcription factor Sox9 (1.33 log2 fold higher). Cell cycle analysis demonstrated that the majority of both progenitor and mature NP cells were in the G1 (resting) phase, and expression of genes related to cell proliferation was modest or absent in both populations (FIG. 22).

TABLE 3
Genes significantly upregulated in mature versus progenitor NP
cells, ranked in descending order by relative log2 fold change.

| Gene | Log2 Fold Change | p (adjusted) |
|---|---|---|
| Fn1 | 4.11 | 4.26E−96 |
| Acan | 4.11 | 1.25E−100 |
| Fos | 3.32 | 2.06E−40 |
| Col2a1 | 3.25 | 2.44E−52 |
| Atf3 | 3.10 | 7.44E−57 |
| Spp1 | 2.98 | 1.89E−87 |
| Egr1 | 2.86 | 1.95E−09 |
| Fosb | 2.74 | 4.22E−61 |
| Junb | 2.69 | 2.01E−29 |
| CD109 | 2.56 | 2.21E−91 |
| Lyst | 2.53 | 5.84E−69 |
| Htra1 | 2.52 | 2.30E−89 |
| Col6a2 | 2.23 | 7.14E−83 |
| Ecrg4 | 2.23 | 5.36E−57 |
| Col6a1 | 2.20 | 6.04E−78 |
| Col11a1 | 2.16 | 3.57E−59 |
| Col6a3 | 2.10 | 1.83E−77 |
| Hapln1 | 2.10 | 1.12E−72 |
| Ccn2 | 2.09 | 2.03E−05 |
| Sparc | 2.05 | 1.52E−48 |
| Ahnak | 2.03 | 3.82E−70 |
| Mgp | 2.02 | 7.88E−38 |
| Susd5 | 2.01 | 1.58E−77 |
| Sostdc1 | 1.99 | 7.86E−43 |
| Emp1 | 1.98 | 7.24E−17 |
| Socs3 | 1.96 | 5.87E−07 |
| CD9 | 1.90 | 1.21E−35 |
| Ccn1 | 1.90 | 5.80E−24 |
| Ptgs1 | 1.89 | 1.89E−66 |
| App | 1.88 | 6.79E−76 |
| Dst | 1.87 | 1.10E−65 |
| Fmod | 1.83 | 3.00E−57 |
| Gadd45g | 1.83 | 7.51E−48 |
| Slc12a2 | 1.80 | 2.93E−53 |
| Mfge8 | 1.71 | 8.43E−50 |
| Ahnak2 | 1.69 | 1.45E−74 |
| Peg3 | 1.65 | 3.89E−50 |
| Cdkn1a | 1.65 | 1.61E−21 |
| Dusp1 | 1.63 | 2.82E−09 |
| Cpe | 1.57 | 3.77E−60 |
| Sparl1 | 1.56 | 5.45E−64 |
| Itm2a | 1.53 | 1.09E−58 |
| Ecm1 | 1.53 | 1.67E−48 |
| Fgl2 | 1.51 | 3.46E−33 |
| Zbtb20 | 1.49 | 2.02E−58 |
| Gm26532 | 1.47 | 9.18E−03 |
| Col11a2 | 1.46 | 6.52E−51 |
| Xylt1 | 1.45 | 4.61E−54 |
| Id1 | 1.43 | 1.27E−43 |
| Rrbp1 | 1.43 | 1.24E−67 |
| Klf4 | 1.43 | 4.97E−16 |
| Mt1 | 1.41 | 9.80E−15 |
| Ddr2 | 1.41 | 4.34E−45 |
| Cp | 1.41 | 4.57E−49 |
| Igfbp7 | 1.40 | 3.81E−55 |
| Sdc4 | 1.39 | 1.45E−49 |
| Snorc | 1.38 | 1.80E−57 |
| Id3 | 1.38 | 7.94E−19 |
| Ptn | 1.37 | 7.39E−42 |
| Meltf | 1.37 | 3.61E−28 |
| Ltbp1 | 1.37 | 2.79E−27 |
| Npnt | 1.35 | 2.94E−62 |
| Ifrd1 | 1.34 | 3.27E−14 |
| Lgmn | 1.33 | 6.40E−64 |
| Meg3 | 1.33 | 1.26E−04 |
| Cck | 1.33 | 7.61E−35 |
| Sox9 | 1.33 | 4.73E−43 |
| Kif21a | 1.32 | 7.25E−54 |
| Scd2 | 1.31 | 3.58E−51 |
| Lrp1 | 1.31 | 7.01E−45 |
| Dsp | 1.30 | 1.53E−54 |
| Ctsl | 1.28 | 1.07E−48 |
| Hspg2 | 1.27 | 1.31E−37 |
| Plod2 | 1.27 | 7.02E−44 |
| Klf9 | 1.26 | 5.81E−11 |
| Itga5 | 1.24 | 4.77E−15 |
| Cytl1 | 1.23 | 1.57E−27 |
| Ehd3 | 1.22 | 2.23E−34 |
| Zim1 | 1.21 | 9.54E−42 |
| Lmo7 | 1.20 | 8.55E−45 |
| Cspg4 | 1.18 | 3.76E−30 |
| Angptl1 | 1.17 | 7.35E−42 |
| Chrm2 | 1.16 | 1.98E−46 |
| Gas1 | 1.14 | 2.91E−39 |
| Fat1 | 1.13 | 1.21E−30 |
| Palld | 1.12 | 5.41E−42 |
| Cdh6 | 1.12 | 4.75E−24 |
| Olfml2b | 1.11 | 1.31E−35 |
| Loxl2 | 1.11 | 5.18E−46 |
| Prnp | 1.11 | 1.41E−53 |
| Col15a1 | 1.10 | 6.28E−11 |
| Serpinh1 | 1.09 | 1.18E−47 |
| Smoc1 | 1.09 | 6.41E−49 |
| Cpm | 1.08 | 1.39E−22 |
| Csgalnact1 | 1.06 | 2.00E−49 |
| Nr4a2 | 1.05 | 3.18E−08 |
| P4ha1 | 1.05 | 1.44E−59 |
| Serpina1e | 1.05 | 5.76E−30 |
| Prelp | 1.04 | 2.22E−42 |
| Tnfrsf12a | 1.03 | 1.11E−28 |
| Ctsb | 1.03 | 1.06E−56 |
| Id2 | 1.02 | 8.34E−28 |
| Tob1 | 1.01 | 8.28E−38 |
| Pde4b | 1.00 | 7.58E−38 |
| Aebp1 | 0.99 | 2.05E−47 |
| Mxra8 | 0.97 | 4.70E−27 |
| Slc15a5 | 0.97 | 1.05E−52 |
| Sgk1 | 0.97 | 1.87E−14 |
| Tubb2a | 0.95 | 5.98E−28 |
| Timp2 | 0.95 | 3.40E−37 |
| Tnfrsf11b | 0.94 | 2.66E−21 |
| Cdh2 | 0.94 | 2.59E−46 |
| Ucma | 0.93 | 9.36E−11 |
| Plcl1 | 0.92 | 4.62E−34 |
| Tln2 | 0.92 | 6.88E−49 |
| Wwp2 | 0.91 | 3.63E−26 |
| Bhlhe40 | 0.91 | 1.03E−08 |
| Itga1 | 0.90 | 1.22E−13 |
| Cpxm2 | 0.90 | 1.73E−34 |
| Limch1 | 0.90 | 1.16E−27 |
| Slc7a2 | 0.90 | 1.49E−43 |
| Enpp2 | 0.88 | 4.95E−07 |
| Bgn | 0.88 | 3.97E−41 |
| Tgfb2 | 0.88 | 1.09E−57 |
| Pcolce2 | 0.87 | 7.08E−49 |
| Pdia6 | 0.87 | 1.63E−45 |
| Gpc3 | 0.87 | 6.55E−43 |
| Tsc22d1 | 0.86 | 2.01E−29 |
| Itih5 | 0.86 | 1.12E−08 |
| Slc2a1 | 0.86 | 3.61E−33 |
| Tnxb | 0.85 | 1.71E−04 |
| Pkd2 | 0.84 | 5.50E−54 |
| Flt1 | 0.84 | 4.80E−22 |
| Cst3 | 0.83 | 9.18E−33 |
| Crip1 | 0.81 | 2.12E−16 |
| Fkbp9 | 0.81 | 7.17E−44 |
| Nrp2 | 0.80 | 7.26E−37 |
| Pcdh7 | 0.80 | 2.99E−24 |
| Trps1 | 0.80 | 4.45E−39 |
| Gja1 | 0.79 | 6.89E−14 |
| Pim3 | 0.78 | 1.21E−49 |
| Col9a3 | 0.77 | 2.58E−21 |
| Creb3l2 | 0.77 | 7.41E−39 |
| Hmgcs1 | 0.76 | 1.93E−18 |
| Gpx3 | 0.76 | 1.86E−07 |
| Gsn | 0.76 | 9.38E−42 |
| Fkbp10 | 0.74 | 8.76E−34 |
| Pmepa1 | 0.73 | 2.12E−16 |
| Cavin2 | 0.73 | 5.61E−33 |
| Sox6 | 0.73 | 1.31E−56 |
| Hbb-bt | 0.73 | 5.42E−04 |
| Hif1a | 0.72 | 9.24E−54 |
| Myh11 | 0.71 | 2.84E−11 |
| Tafa1 | 0.70 | 1.33E−12 |
| Lmna | 0.69 | 2.63E−24 |
| S100b | 0.69 | 8.47E−21 |
| P3h4 | 0.68 | 4.54E−27 |
| Atp6v0c | 0.68 | 6.50E−60 |
| Rnase4 | 0.68 | 8.65E−39 |
| Ptprd | 0.67 | 1.23E−18 |
| Pmp22 | 0.67 | 9.13E−39 |
| Lifr | 0.66 | 7.68E−47 |
| Hspa1a | 0.66 | 2.16E−18 |
| Sulf2 | 0.65 | 4.82E−18 |
| Plxna2 | 0.65 | 8.87E−52 |
| Cdh13 | 0.65 | 3.07E−28 |
| Fxyd3 | 0.65 | 1.84E−27 |
| Auts2 | 0.64 | 3.40E−44 |
| Cdon | 0.64 | 1.45E−21 |
| Hopx | 0.63 | 4.65E−12 |
| Cd302 | 0.63 | 3.24E−35 |
| Serpine1 | 0.62 | 2.94E−14 |
| Tm4sf1 | 0.61 | 6.15E−12 |
| Krt18 | 0.61 | 5.20E−11 |
| Itgb5 | 0.61 | 7.36E−10 |
| S100a10 | 0.61 | 1.87E−12 |
| Ccdc80 | 0.61 | 5.99E−10 |
| Col1a1 | 0.61 | 8.39E−07 |
| Mpzl2 | 0.59 | 7.47E−08 |
| Fndc3b | 0.59 | 3.33E−25 |
| Hba-a2 | 0.58 | 1.88E−06 |
| Fermt2 | 0.58 | 2.21E−24 |
| Apoe | 0.57 | 2.44E−02 |
| Gpc6 | 0.57 | 1.60E−15 |
| Abi3bp | 0.57 | 2.10E−48 |
| Nfatc2 | 0.57 | 1.79E−48 |
| Rcn3 | 0.56 | 2.14E−25 |
| Calr | 0.56 | 2.64E−32 |
| Timp3 | 0.56 | 3.49E−32 |
| Fgfr1 | 0.55 | 1.37E−34 |
| Tmx4 | 0.54 | 7.10E−65 |
| Tgfbr3 | 0.54 | 4.15E−06 |
| Tmem255a | 0.54 | 1.95E−30 |
| Adcy5 | 0.54 | 6.11E−33 |
| Pde1a | 0.54 | 5.05E−13 |
| Pcp4l1 | 0.53 | 3.54E−23 |
| Ikbip | 0.53 | 6.06E−20 |
| Htra3 | 0.52 | 1.23E−36 |
| Crip2 | 0.52 | 4.05E−18 |
| Gpx8 | 0.51 | 8.66E−21 |
| Tspan3 | 0.51 | 3.29E−25 |
| Rgcc | 0.51 | 1.93E−02 |
| Slc26a2 | 0.51 | 2.07E−02 |
| Ctsk | 0.51 | 1.42E−06 |
| Slc20a1 | 0.50 | 1.08E−19 |
| P4ha2 | 0.49 | 8.05E−04 |
| Lypd1 | 0.48 | 3.68E−03 |
| P3h2 | 0.48 | 1.09E−35 |
| Srgn | 0.47 | 1.10E−20 |
| Gm26802 | 0.46 | 4.84E−11 |
| Itm2c | 0.46 | 6.99E−17 |
| Dnajb1 | 0.46 | 1.14E−02 |
| Btbd3 | 0.45 | 1.11E−21 |
| S100a3 | 0.45 | 2.91E−37 |
| Chst11 | 0.44 | 9.43E−30 |
| Ltbp3 | 0.44 | 9.48E−22 |
| Crtap | 0.43 | 1.05E−17 |
| Plaur | 0.43 | 3.53E−04 |
| Sema3c | 0.43 | 7.43E−15 |
| Socs1 | 0.43 | 1.32E−34 |
| Grb10 | 0.43 | 7.72E−29 |
| Gadd45b | 0.43 | 3.05E−13 |
| Myc | 0.43 | 8.88E−08 |
| Scara3 | 0.41 | 1.34E−40 |
| Icam1 | 0.41 | 1.18E−19 |
| Fgfr3 | 0.41 | 7.53E−10 |
| Gpc1 | 0.40 | 1.08E−23 |
| Ppic | 0.39 | 4.66E−02 |
| Sgms2 | 0.39 | 3.42E−20 |
| Sbsn | 0.38 | 4.61E−07 |
| Ptdss2 | 0.38 | 6.57E−63 |
| Maged2 | 0.37 | 1.05E−13 |
| Adamts2 | 0.37 | 7.06E−06 |
| Fkbp14 | 0.37 | 1.23E−23 |
| Cav2 | 0.37 | 7.52E−19 |
| Azgp1 | 0.36 | 6.10E−22 |
| Srpx2 | 0.36 | 5.08E−07 |
| C1galt1c1 | 0.36 | 5.80E−03 |
| Mageh1 | 0.35 | 9.09E−22 |
| Clu | 0.35 | 1.17E−13 |
| Npdc1 | 0.34 | 2.29E−27 |
| Tmod2 | 0.34 | 3.06E−12 |
| Plpp1 | 0.34 | 1.26E−08 |
| Slit3 | 0.34 | 4.42E−13 |
| Nfix | 0.34 | 9.53E−19 |
| Tmem176b | 0.33 | 2.04E−15 |
| Lamb2 | 0.33 | 1.91E−04 |
| Bhlhe41 | 0.32 | 6.47E−03 |
| Sobp | 0.32 | 4.57E−39 |
| Ptgds | 0.32 | 5.71E−44 |
| Raph1 | 0.31 | 9.85E−30 |
| Kdelr3 | 0.31 | 1.76E−03 |
| Selenbp1 | 0.30 | 7.05E−13 |
| Sfrp5 | 0.30 | 1.24E−22 |
| Ccn5 | 0.29 | 4.60E−04 |
| Maf | 0.29 | 7.09E−11 |
| Ifi27l2a | 0.29 | 3.81E−02 |
| Id4 | 0.29 | 1.11E−02 |
| 9330158H04Rik | 0.29 | 3.95E−21 |
| Lmcd1 | 0.29 | 8.36E−43 |
| Aspa | 0.28 | 1.15E−27 |
| Fgfr2 | 0.28 | 2.60E−35 |
| Pak3 | 0.28 | 3.72E−45 |
| Itpkc | 0.28 | 1.20E−29 |
| Gfod2 | 0.28 | 2.10E−75 |
| Rnf149 | 0.27 | 5.74E−55 |
| Thbs3 | 0.26 | 1.63E−07 |
| Rhoj | 0.26 | 3.49E−10 |
| Efemp2 | 0.26 | 1.68E−23 |
| Bmp1 | 0.25 | 3.58E−56 |
| Cxcl2 | 0.25 | 1.18E−16 |
| Irx5 | 0.25 | 2.76E−06 |
| Slurp1 | 0.25 | 1.86E−31 |
| Egr4 | 0.25 | 2.48E−35 |

TABLE 4
Genes significantly downregulated in mature versus progenitor NP
cells, ranked in descending order by relative log2 fold change.

| Gene | Log2 Fold Change | p (adjusted) |
|---|---|---|
| Rbp4 | −1.63 | 2.50E−18 |
| Cdo1 | −1.35 | 5.85E−07 |
| Plp1 | −1.18 | 2.27E−37 |
| Cox8b | −0.99 | 6.77E−61 |
| 3110079O15Rik | −0.92 | 2.41E−02 |
| Hagh | −0.80 | 1.00E−04 |
| Mif | −0.70 | 8.47E−18 |
| Npm1 | −0.70 | 1.96E−04 |
| Mt3 | −0.69 | 2.06E−66 |
| 1500011K16Rik | −0.69 | 2.54E−04 |
| Glrx5 | −0.63 | 2.00E−15 |
| Rbp1 | −0.62 | 1.09E−13 |
| Car3 | −0.61 | 6.69E−12 |
| Plac8 | −0.61 | 5.76E−08 |
| Myl9 | −0.57 | 7.17E−22 |
| Fabp5 | −0.56 | 2.90E−18 |
| Blvrb | −0.55 | 1.27E−06 |
| 1500015O10Rik | −0.55 | 3.40E−13 |
| Nhp2 | −0.53 | 1.17E−06 |
| Fxyd6 | −0.51 | 3.74E−03 |
| Hebp1 | −0.51 | 1.62E−08 |
| Car2 | −0.49 | 8.35E−11 |
| Sln | −0.42 | 6.51E−08 |
| Vat1 | −0.40 | 6.75E−03 |
| Car1 | −0.40 | 1.39E−52 |
| 1110008F13Rik | −0.36 | 9.73E−07 |
| Fkbp4 | −0.36 | 3.75E−04 |
| Col22a1 | −0.36 | 2.71E−20 |
| Ndufa4l2 | −0.35 | 8.70E−03 |
| Nkg7 | −0.35 | 9.85E−60 |
| Ncl | −0.32 | 9.08E−04 |
| Wfdc21 | −0.31 | 5.92E−12 |
| Timm8a1 | −0.30 | 5.03E−09 |
| Pla2g12a | −0.29 | 3.11E−07 |
| Ctse | −0.28 | 8.89E−70 |
| Mustn1 | −0.28 | 1.53E−21 |
| Apex1 | −0.27 | 1.13E−03 |
| Isg20 | −0.27 | 6.93E−03 |
| Clec12a | −0.27 | 1.98E−29 |
| Ifrd2 | −0.26 | 1.25E−46 |
| Hist1h4i | −0.26 | 3.27E−07 |
| Col1a2 | −0.25 | 5.02E−08 |
| Stard10 | −0.25 | 3.31E−48 |

### Pathway Analysis

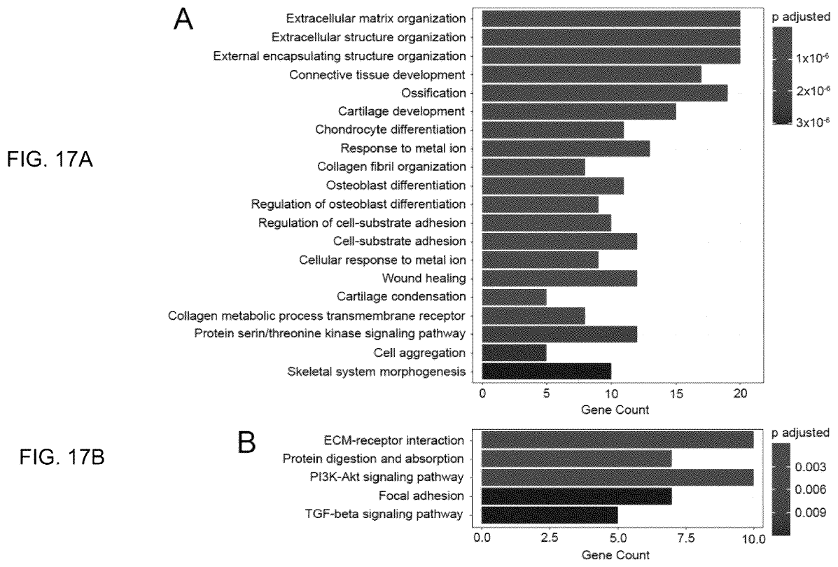

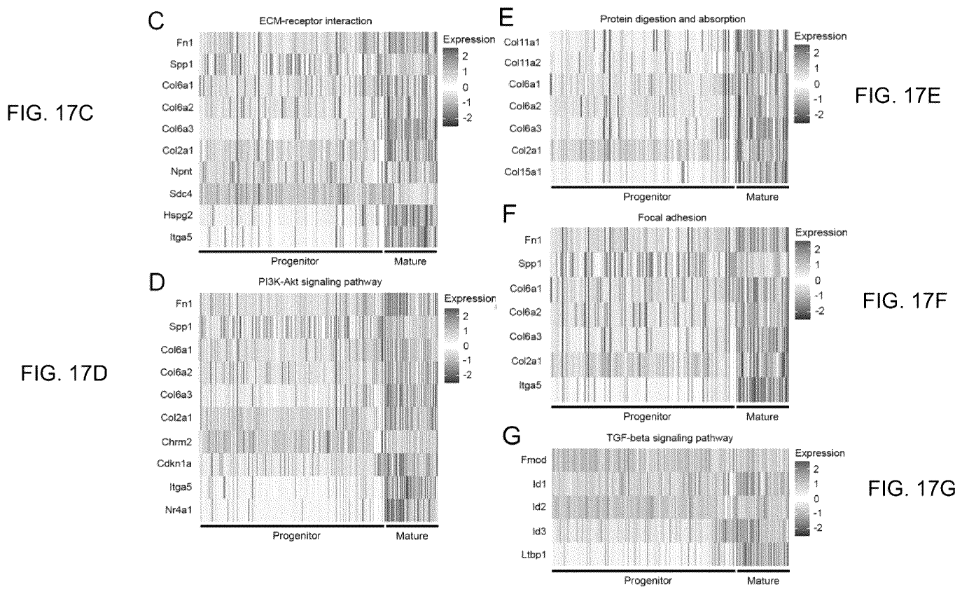

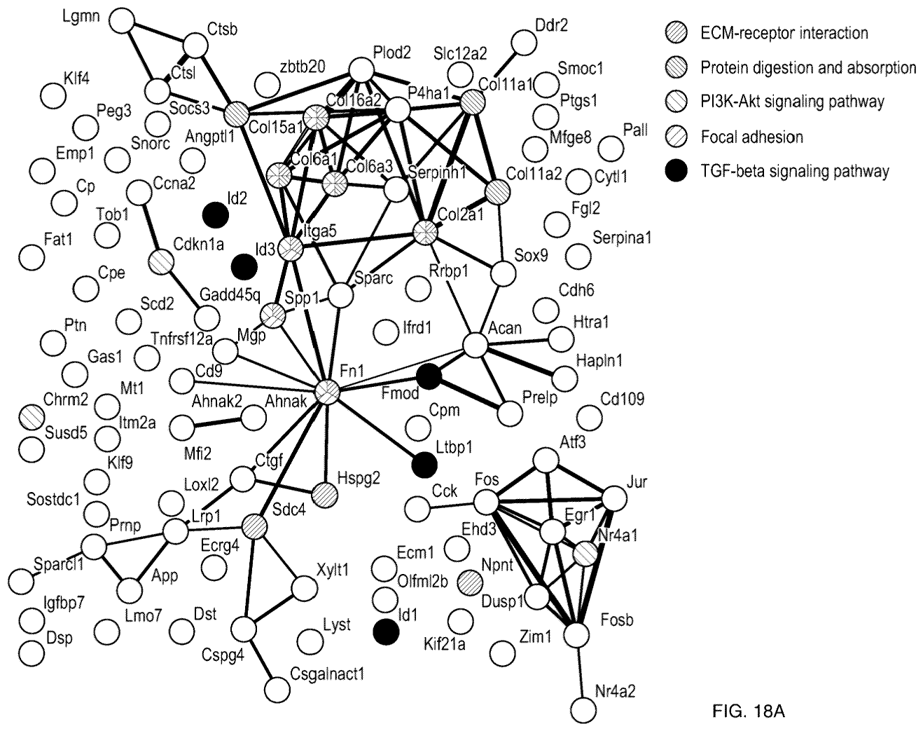

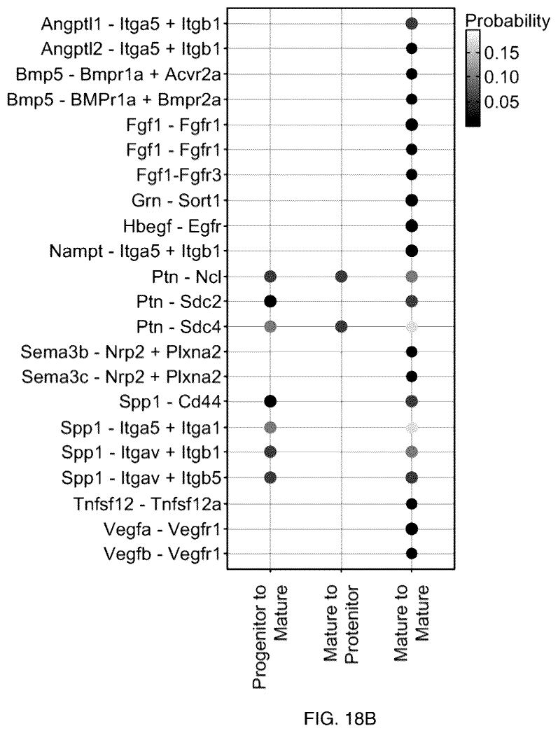

To provide mechanistic insights into the relationship between mature and progenitor NP cells, GO, KEGG, and PPI network analyses was performed. Top GO terms enriched in mature versus progenitor NP cells included those related to ECM organization, connective tissue development, and chondrocyte differentiation (FIG. 17A). With respect to KEGG analysis, 5 pathways were identified that were significantly enriched in mature versus progenitor NP cells: ECM-receptor interaction, protein digestion and absorption, PI3K-Akt signaling, focal adhesion, and transforming growth factor-beta (TGF-β) signaling (FIGS. 17B-G). Several of these pathways exhibited common elements, including collagens II and VI, osteopontin (Spp1) and fibronectin-1 (Fn1), which were expressed at significantly higher levels in mature vs progenitor NP cells. PPI network analysis demonstrated significant interactions between elements of these pathways (FIG. 18A). Genes with the greatest (>5) number of interactions included Fn1, Col6a1, Col6a2, Acan, Col2a1, collagen XI-alpha 1 (Col11a1), Fosb and early growth response factor 1 (Egr1). Using the CellChat tool (Jin, S, et al. Nat Commun 2021; 12:1088), a total of 93 interactions between and within progenitor and mature NP cells were identified (FIG. 18B and Table 5). Interactions categories included secreted signaling, ECM-receptor and cell-cell contact. With respect to secreted signaling, there were 7 ligand-receptor interactions from progenitor to mature stage NP cells, 2 from mature to progenitor NP cells, and 22 from mature to mature NP cells (FIG. 18B). The secreted signaling interactions from progenitor to mature NP cells involved either pleiotrophin (Ptn) or Spp1.

TABLE 5
Cell-cell interactions identified using CellChat.

| | | | | | | Interaction | |
|---|---|---|---|---|---|---|---|
| Source | Target | Annotation | Ligand | Receptor | Probability | Name | Evidence |
| Progenitor NP cells | Mature NP cells | Secreted Signaling | Ptn | Sdc2 | 0.007 | Ptn − Sdc2 | PMID: 28356350; |
| | | | | | | | PMID: 25620911 |
| Progenitor NP cells | Mature NP cells | Secreted Signaling | Ptn | Sdc4 | 0.096 | Ptn − Sdc4 | PMID: 28356350; |
| | | | | | | | PMID: 25620911 |
| Progenitor NP cells | Mature NP cells | Secreted Signaling | Ptn | Ncl | 0.051 | Ptn − Ncl | PMID: 28356350; |
| | | | | | | | PMID: 25620911 |
| Progenitor NP cells | Mature NP cells | Secreted Signaling | Spp1 | Cd44 | 0.011 | Spp1 − Cd44 | PMID: 21907263 |
| Progenitor NP cells | Mature NP cells | Secreted Signaling | Spp1 | Itgav + Itgb1 | 0.054 | Spp1 − (Itgav + | PMID: 21907263 |
| | | | | | | Itgb1) | |
| Progenitor NP cells | Mature NP cells | Secreted Signaling | Spp1 | Itgav + Itgb5 | 0.028 | Spp1 − (Itgav + | PMID: 21907263 |
| | | | | | | Itgb5) | |
| Progenitor NP cells | Mature NP cells | Secreted Signaling | Spp1 | Itga5 + Itgb1 | 0.092 | Spp1 − (Itga5 + | PMID: 21907263 |
| | | | | | | Itgb1) | |
| Mature NP cells | Progenitor NP cells | ECM-Receptor | Col1a1 | Sdc4 | 0.009 | Col1a1 − Sdc4 | KEGG: mmu04512 |
| Mature NP cells | Progenitor NP cells | ECM-Receptor | Col1a2 | Sdc4 | 0.002 | Col1a2 − Sdc4 | KEGG: mmu04512 |
| Mature NP cells | Progenitor NP cells | ECM-Receptor | Col2a1 | Sdc4 | 0.026 | Col2a1 − Sdc4 | KEGG: mmu04512 |
| Mature NP cells | Progenitor NP cells | ECM-Receptor | Col6a1 | Sdc4 | 0.028 | Col6a1 − Sdc4 | KEGG: mmu04512 |
| Mature NP cells | Progenitor NP cells | ECM-Receptor | Col6a2 | Sdc4 | 0.025 | Col6a2 − Sdc4 | KEGG: mmu04512 |
| Mature NP cells | Progenitor NP cells | ECM-Receptor | Col6a3 | Sdc4 | 0.02 | Col6a3 − Sdc4 | KEGG: mmu04512 |
| Mature NP cells | Progenitor NP cells | ECM-Receptor | Col9a3 | Sdc4 | 0.003 | Col9a3 − Sdc4 | KEGG: mmu04512 |
| Mature NP cells | Progenitor NP cells | ECM-Receptor | Fn1 | Sdc4 | 0.048 | Fn1 − Sdc4 | KEGG: mmu04512 |
| Mature NP cells | Progenitor NP cells | ECM-Receptor | Thbs3 | Sdc4 | 0.001 | Thbs3 − Sdc4 | KEGG: mmu04512 |
| Mature NP cells | Progenitor NP cells | ECM-Receptor | Tnxb | Sdc4 | 0.008 | Tnxb − Sdc4 | KEGG: mmu04512 |
| Mature NP cells | Progenitor NP cells | Secreted Signaling | Ptn | Sdc4 | 0.037 | Ptn − Sdc4 | PMID: 28356350; |
| | | | | | | | PMID: 25620911 |
| Mature NP cells | Progenitor NP cells | Secreted Signaling | Ptn | Ncl | 0.035 | Ptn − Ncl | PMID: 28356350; |
| | | | | | | | PMID: 25620911 |
| Mature NP cells | Mature NP cells | Cell-Cell Contact | Cdh2 | Cdh2 | 0.048 | Cdh2 − Cdh2 | KEGG: mmu04514 |
| Mature NP cells | Mature NP cells | Cell-Cell Contact | Dsg2 | Dsc3 | 0.001 | Dsg2 − Dsc3 | PMID: 27298358 |
| Mature NP cells | Mature NP cells | Cell-Cell Contact | Ncam1 | Fgfr1 | 0.004 | Ncam1 − Fgfr1 | PMID: 12791257 |
| Mature NP cells | Mature NP cells | Cell-Cell Contact | Ncam1 | Ncam1 | 0.013 | Ncam1 − Ncam1 | KEGG: mmu04514 |
| Mature NP cells | Mature NP cells | ECM-Receptor | Col1a1 | Itga1 + Itgb1 | 0.025 | Col1a1 − (Itga1 + | KEGG: mmu04512 |
| | | | | | | Itgb1) | |
| Mature NP cells | Mature NP cells | ECM-Receptor | Col1a1 | Itga10 + Itgb1 | 0.005 | Col1a1 − (Itga10 + | KEGG: mmu04512 |
| | | | | | | Itgb1) | |
| Mature NP cells | Mature NP cells | ECM-Receptor | Col1a1 | Cd44 | 0.003 | Col1a1 − Cd44 | KEGG: mmu04512 |
| Mature NP cells | Mature NP cells | ECM-Receptor | Col1a1 | Sdc4 | 0.055 | Col1a1 − Sdc4 | KEGG: mmu04512 |
| Mature NP cells | Mature NP cells | ECM-Receptor | Col1a2 | Itga1 + Itgb1 | 0.005 | Col1a2 − (Itga1 + | KEGG: mmu04512 |
| | | | | | | Itgb1) | |
| Mature NP cells | Mature NP cells | ECM-Receptor | Col1a2 | Itga10 + Itgb1 | 0.001 | Col1a2 − (Itga10 + | KEGG: mmu04512 |
| | | | | | | Itgb1) | |
| Mature NP cells | Mature NP cells | ECM-Receptor | Col1a2 | Cd44 | 0.001 | Col1a2 − Cd44 | KEGG: mmu04512 |
| Mature NP cells | Mature NP cells | ECM-Receptor | Col1a2 | Sdc4 | 0.012 | Col1a2 − Sdc4 | KEGG: mmu04512 |
| Mature NP cells | Mature NP cells | ECM-Receptor | Col2a1 | Itga1 + Itgb1 | 0.068 | Col2a1 − (Itga1 + | KEGG: mmu04512 |
| | | | | | | Itgb1) | |
| Mature NP cells | Mature NP cells | ECM-Receptor | Col2a1 | Itga10 + Itgb1 | 0.013 | Col2a1 − (Itga10 + | KEGG: mmu04512 |
| | | | | | | Itgb1) | |
| Mature NP cells | Mature NP cells | ECM-Receptor | Col2a1 | Cd44 | 0.008 | Col2a1 − Cd44 | KEGG: mmu04512 |
| Mature NP cells | Mature NP cells | ECM-Receptor | Col2a1 | Sdc4 | 0.143 | Col2a1 − Sdc4 | KEGG: mmu04512 |
| Mature NP cells | Mature NP cells | ECM-Receptor | Col6a1 | Itga1 + Itgb1 | 0.074 | Col6a1 − (Itga1 + | KEGG: mmu04512 |
| | | | | | | Itgb1) | |
| Mature NP cells | Mature NP cells | ECM-Receptor | Col6a1 | Itga10 + Itgb1 | 0.014 | Col6a1 − (Itga10 + | KEGG: mmu04512 |
| | | | | | | Itgb1) | |
| Mature NP cells | Mature NP cells | ECM-Receptor | Col6a1 | Cd44 | 0.008 | Col6a1 − Cd44 | KEGG: mmu04512 |
| Mature NP cells | Mature NP cells | ECM-Receptor | Col6a1 | Sdc4 | 0.154 | Col6a1 − Sdc4 | KEGG: mmu04512 |
| Mature NP cells | Mature NP cells | ECM-Receptor | Col6a2 | Itga1 + Itgb1 | 0.066 | Col6a2 − (Itga1 + | KEGG: mmu04512 |
| | | | | | | Itgb1) | |
| Mature NP cells | Mature NP cells | ECM-Receptor | Col6a2 | Itga10 + Itgb1 | 0.013 | Col6a2 − (Itga10 + | KEGG: mmu04512 |
| | | | | | | Itgb1) | |
| Mature NP cells | Mature NP cells | ECM-Receptor | Col6a2 | Cd44 | 0.007 | Col6a2 − Cd44 | KEGG: mmu04512 |
| Mature NP cells | Mature NP cells | ECM-Receptor | Col6a2 | Sdc4 | 0.14 | Col6a2 − Sdc4 | KEGG: mmu04512 |
| Mature NP cells | Mature NP cells | ECM-Receptor | Col6a3 | Itga1 + Itgb1 | 0.053 | Col6a3 − (Itga1 + | KEGG: mmu04512 |
| | | | | | | Itgb1) | |
| Mature NP cells | Mature NP cells | ECM-Receptor | Col6a3 | Itga10 + Itgb1 | 0.01 | Col6a3 − (Itga10 + | KEGG: mmu04512 |
| | | | | | | Itgb1) | |
| Mature NP cells | Mature NP cells | ECM-Receptor | Col6a3 | Cd44 | 0.006 | Col6a3 − Cd44 | KEGG: mmu04512 |
| Mature NP cells | Mature NP cells | ECM-Receptor | Col6a3 | Sdc4 | 0.113 | Col6a3 − Sdc4 | KEGG: mmu04512 |
| Mature NP cells | Mature NP cells | ECM-Receptor | Col9a3 | Itga1 + Itgb1 | 0.009 | Col9a3 − (Itga1 + | KEGG: mmu04512 |
| | | | | | | Itgb1) | |
| Mature NP cells | Mature NP cells | ECM-Receptor | Col9a3 | Itga10 + Itgb1 | 0.002 | Col9a3 − (Itga10 + | KEGG: mmu04512 |
| | | | | | | Itgb1) | |
| Mature NP cells | Mature NP cells | ECM-Receptor | Co19a3 | Cd44 | 0.001 | Col9a3 − Cd44 | KEGG: mmu04512 |
| Mature NP cells | Mature NP cells | ECM-Receptor | Col9a3 | Sdc4 | 0.02 | Col9a3 − Sdc4 | KEGG: mmu04512 |
| Mature NP cells | Mature NP cells | ECM-Receptor | Fn1 | Itga5 + Itgb1 | 0.12 | Fn1 − (Itga5 + | KEGG: mmu04512 |
| | | | | | | Itgb1) | |
| Mature NP cells | Mature NP cells | ECM-Receptor | Fn1 | Itgav + Itgb1 | 0.072 | Fn1 − (Itgav + | KEGG: mmu04512 |
| | | | | | | Itgb1) | |
| Mature NP cells | Mature NP cells | ECM-Receptor | Fn1 | Cd44 | 0.014 | Fn1 − Cd44 | KEGG: mmu04512 |
| Mature NP cells | Mature NP cells | ECM-Receptor | Fn1 | Sdc4 | 0.239 | Fn1 − Sdc4 | KEGG: mmu04512 |
| Mature NP cells | Mature NP cells | ECM-Receptor | Hspg2 | Dag1 | 0.019 | Hspg2 − Dag1 | KEGG: mmu04512 |
| Mature NP cells | Mature NP cells | ECM-Receptor | Lama4 | Itga1 + Itgb1 | 0.005 | Lama4 − (Itga1 + | KEGG: mmu04512 |
| | | | | | | Itgb1) | |
| Mature NP cells | Mature NP cells | ECM-Receptor | Lama4 | Cd44 | 0.001 | Lama4 − Cd44 | KEGG: mmu04512 |
| Mature NP cells | Mature NP cells | ECM-Receptor | Lama4 | Dag1 | 0.003 | Lama4 − Dag1 | KEGG: mmu04512 |
| Mature NP cells | Mature NP cells | ECM-Receptor | Lamb2 | Itga1 + Itgb1 | 0.004 | Lamb2 − (Itga1 + | KEGG: mmu04512 |
| | | | | | | Itgb1) | |
| Mature NP cells | Mature NP cells | ECM-Receptor | Lamb2 | Cd44 | 0 | Lamb2 − Cd44 | KEGG: mmu04512 |
| Mature NP cells | Mature NP cells | ECM-Receptor | Lamb2 | Dag1 | 0.002 | Lamb2 − Dag1 | KEGG: mmu04512 |
| Mature NP cells | Mature NP cells | ECM-Receptor | Lamb3 | Itga1 + Itgb1 | 0.02 | Lamb3 − (Itga1 + | KEGG: mmu04512 |
| | | | | | | Itgb1) | |
| Mature NP cells | Mature NP cells | ECM-Receptor | Lamb3 | Cd44 | 0.002 | Lamb3 − Cd44 | KEGG: mmu04512 |
| Mature NP cells | Mature NP cells | ECM-Receptor | Lamb3 | Dag1 | 0.011 | Lamb3 − Dag1 | KEGG: mmu04512 |
| Mature NP cells | Mature NP cells | ECM-Receptor | Lamc1 | Itga1 + Itgb1 | 0.005 | Lamc1 − (Itga1 + | KEGG: mmu04512 |
| | | | | | | Itgb1) | |
| Mature NP cells | Mature NP cells | ECM-Receptor | Lamc1 | Cd44 | 0.001 | Lamc1 − Cd44 | KEGG: mmu04512 |
| Mature NP cells | Mature NP cells | ECM-Receptor | Lamc1 | Dag1 | 0.003 | Lamc1 − Dag1 | KEGG: mmu04512 |
| Mature NP cells | Mature NP cells | ECM-Receptor | Thbs3 | Sdc4 | 0.009 | Thbs3 − Sdc4 | KEGG: mmu04512 |
| Mature NP cells | Mature NP cells | ECM-Receptor | Thbs3 | Cd47 | 0.003 | Thbs3 − Cd47 | KEGG: mmu04512 |
| Mature NP cells | Mature NP cells | ECM-Receptor | Tnxb | Sdc4 | 0.048 | Tnxb − Sdc4 | KEGG: mmu04512 |
| Mature NP cells | Mature NP cells | Secreted Signaling | Angptl1 | Itga1 + Itgb1 | 0.056 | Angptl1 − (Itga1 + | PMID: 24478758 |
| | | | | | | Itgb1) | |
| Mature NP cells | Mature NP cells | Secreted Signaling | Angptl2 | Itga5 + Itgb1 | 0.004 | Angptl2 − (Itga5 + | PMID: 24478758 |
| | | | | | | Itgb1) | |
| Mature NP cells | Mature NP cells | Secreted Signaling | Bmp5 | Bmpr1a + Acvr2a | 0.001 | Bmp5 − (Bmpr1a + | KEGG: mmu04350; |
| | | | | | | Acvr2a) | PMID: 26893264 |
| Mature NP cells | Mature NP cells | Secreted Signaling | Bmp5 | Bmpr1a + Bmpr2 | 0 | Bmp5 − (Bmpr1a + | KEGG: mmu04350; |
| | | | | | | Bmpr2) | PMID: 26893264 |
| Mature NP cells | Mature NP cells | Secreted Signaling | Fgf1 | Fgfr1 | 0.003 | Fgf1 − Fgfr1 | PMC: 4393358 |
| Mature NP cells | Mature NP cells | Secreted Signaling | Fgf1 | Fgfr2 | 0.002 | Fgf1 − Fgfr2 | PMC: 4393358 |
| Mature NP cells | Mature NP cells | Secreted Signaling | Fgf1 | Fgfr3 | 0.003 | Fgf1 − Fgfr3 | PMC: 4393358 |
| Mature NP cells | Mature NP cells | Secreted Signaling | Grn | Sort1 | 0.002 | Grn − Sort1 | PMID: 29555433 |
| Mature NP cells | Mature NP cells | Secreted Signaling | Hbegf | Egfr | 0.001 | Hbegf − Egfr | KEGG: mmu04012 |
| Mature NP cells | Mature NP cells | Secreted Signaling | Nampt | Itga5 + Itgb1 | 0.003 | Nampt − (Itga5 + | PMID: 28490838 |
| | | | | | | Itgb1) | |
| Mature NP cells | Mature NP cells | Secreted Signaling | Ptn | Sdc2 | 0.015 | Ptn − Sdc2 | PMID: 28356350; |
| | | | | | | | PMID: 25620911 |
| Mature NP cells | Mature NP cells | Secreted Signaling | Ptn | Sdc4 | 0.193 | Ptn − Sdc4 | PMID: 28356350; |
| | | | | | | | PMID: 25620911 |
| Mature NP cells | Mature NP cells | Secreted Signaling | Ptn | Ncl | 0.108 | Ptn − Ncl | PMID: 28356350; |
| | | | | | | | PMID: 25620911 |
| Mature NP cells | Mature NP cells | Secreted Signaling | Sema3b | Nrp2 + Plxna2 | 0.002 | Sema3b − (Nrp2 + | PMID: 27533782 |
| | | | | | | Plxna2) | |
| Mature NP cells | Mature NP cells | Secreted Signaling | Sema3C | Nrp2 + Plxna2 | 0.002 | Sema3c − (Nrp2 + | PMID: 27533782 |
| | | | | | | Plxna2) | |
| Mature NP cells | Mature NP cells | Secreted Signaling | Spp1 | Cd44 | 0.018 | Spp1 − Cd44 | PMID: 21907263 |
| Mature NP cells | Mature NP cells | Secreted Signaling | Spp1 | Itgav + Itgb1 | 0.09 | Spp1 − (Itgav + | PMID: 21907263 |
| | | | | | | Itgb1) | |
| Mature NP cells | Mature NP cells | Secreted Signaling | Spp1 | Itgav + Itgb5 | 0.047 | Spp1 − (Itgav + | PMID: 21907263 |
| | | | | | | Itgb5) | |
| Mature NP cells | Mature NP cells | Secreted Signaling | Spp1 | Itga5 + Itgb1 | 0.149 | Spp1 − (Itga5 + | PMID: 21907263 |
| | | | | | | Itgb1) | |
| Mature NP cells | Mature NP cells | Secreted Signaling | Tnfsf12 | Tnfrsf12a | 0.003 | Tnfsf12 − Tnfrsf12a | KEGG: mmu04060 |
| Mature NP cells | Mature NP cells | Secreted Signaling | Vegfa | Flt1 | 0.004 | Vegfa − Vegfr1 | KEGG: mmu04370; |
| | | | | | | | PMID: 16633338 |
| Mature NP cells | Mature NP cells | Secreted Signaling | Vegfb | Flt1 | 0.001 | Vegfb − Vegfr1 | KEGG: mmu04370; |
| | | | | | | | PMID: 16633338 |

### Surface Marker Expression and Spatial Localization of NP Cell Subpopulations

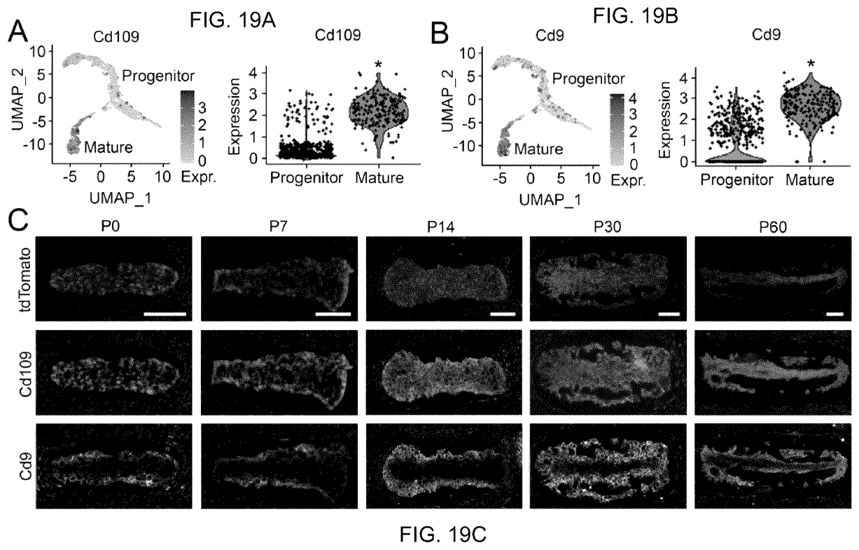

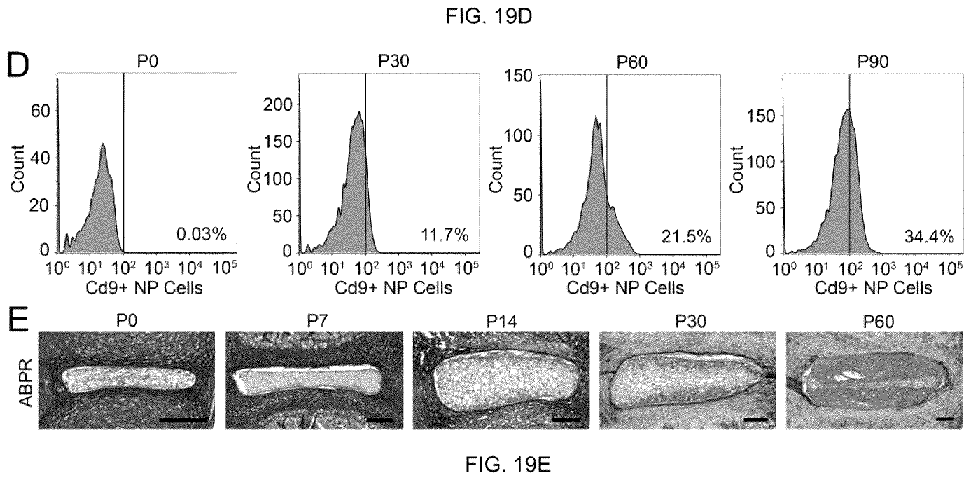

To facilitate in situ spatial localization of distinct NP cell subpopulations within postnatal mouse discs, two candidate surface markers were identified in the single cell sequencing results. Cd109 and Cd9, that exhibited significantly higher expression levels in mature versus progenitor NP cells (2.56 and 1.90-log2 fold higher, respectively; FIGS. 19A and B, and Table 3). Immunofluorescent histology demonstrated that Cd109 protein expression was relatively homogeneous throughout the entire NP at all ages examined, with staining at the periphery being marginally more intense (FIG. 19C). In contrast, spatially heterogenous protein expression was striking for Cd, with immunopositive cells being localized to the periphery of the NP (FIG. 19C). Subjectively, the relative number of these cells became progressively greater with increasing age from P0 to P60. This observation was confirmed using flow cytometry and double immunolabeling for Krt19 and Cd9, which demonstrated that the number of Cd9+ NP cells as a percentage of total NP cells did indeed increase with increasing age, from less than 1% at P0 to more than 30% at P90 (FIG. 19D). Next, ECM elaboration was examined during postnatal growth using Alcian blue (for GAG) and picrosirius red (for collagen) staining (FIG. 19E). Glycosaminoglycan deposition was spatially heterogenous, appearing as a halo surrounding a central cluster of cells that increased in size with increasing age and mirroring the emergence of Cd9+ NP cells (FIG. 19E).

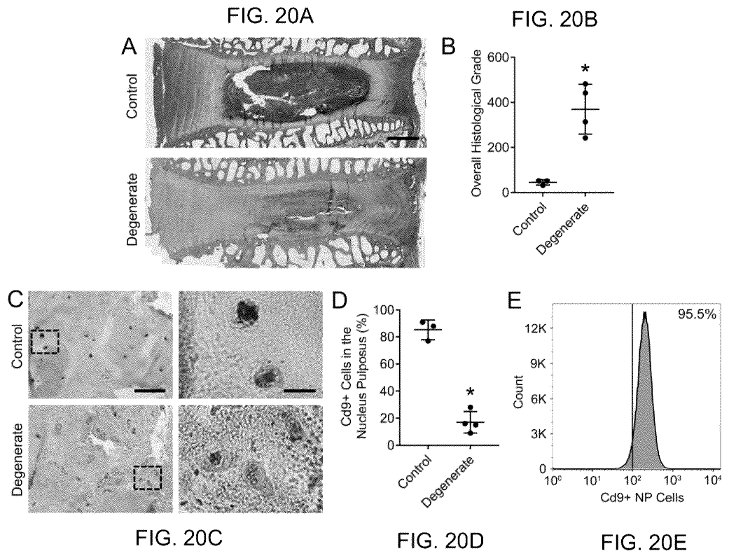

To further investigate whether Cd9 was a marker of the healthy, mature NP cell phenotype, a goat model of moderate severity disc degeneration was used. In this model, 1 U of the enzyme chondroitinase ABC was injected into the NP of goat lumbar discs, resulting in moderate severity degeneration after 12 weeks (FIG. 20A). Successful induction of degeneration was confirmed postmortem via semi-quantitative histological grading (FIG. 20B). Using immunohistochemistry, the presence of Cd9+ NP cells was confirmed in both healthy and degenerate goat discs (FIG. 20C); however, the number of Cd9+ cells as a percentage of total NP cells was significantly lower in degenerate discs (FIG. 20D). Flow cytometry analysis of NP cells from adult healthy goat discs confirmed that the majority of these cells (95.5%) were Cd9+ (FIG. 620E).

The notochordal origin of NP cells is unique amongst skeletal cell types. The notochord is a rod-like structure that is present embryonically in chordates, and functions as a source of long-range morphogens that regulate patterning of the axial skeleton (Smith, L J, et al. Dis Model Mech 2011; 4:31-41). In mice, beginning at around embryonic day E12.5, the notochord undergoes a transformation, contracting within the regions of the future vertebrae, and expanding within the regions of the future discs, to ultimately form the NPs (Smith, L J, et al. Dis Model Mech 2011; 4:31-41). This process occurs similarly across other vertebrates, including humans. The notochordal origin of NP cells in the mouse disc throughout postnatal growth and adulthood has been confirmed though fate-mapping studies that have leveraged notochordal markers such as Shh, 13 Noto, 15 and Foxa2.35 A recent study also showed that NP cells express the cytoskeletal marker Krt19 throughout life (Mohanty, S, et al. Aging Cell 2019; 18:e13006), confirmed in the current study, and leveraged expression of Krt19 and other NP-specific markers such as Krt8 and 18, and brachyury (T) to identify NP cells within the overall sequenced cell population.

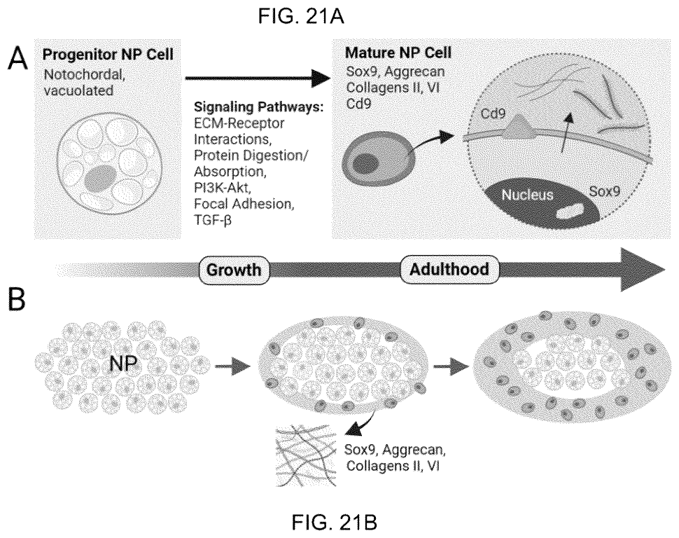

The eventual depletion of progenitor NP cells (historically referred to as “notochordal” cells) in other species, including humans, occurs for reasons still not well understood, and may be due to both longer life span and species-specific physical and biochemical microenvironmental factors. This loss of notochordal NP cells in humans is considered to be a predisposing factor in the initiation and progression of disc degeneration later in life, as notochordal NP cells and their secretome have been shown to be pro-regenerative properties, enhancing proliferation and ECM production, and reducing inflammation (Bach, F C, et al. Front Cell Dev Biol 2021; 9:780749; and Aguiar, D J, et al. Exp Cell Res 1999; 246: 129-137). In this study, scRNA-seq was used to demonstrate emergent heterogeneity amongst notochord-derived NP cells in the postnatal mouse disc (shown schematically in FIG. 21). Specifically, the existence of progenitor and mature NP cells, corresponding to “notochordal” and “chondrocyte-like” NP cells, respectively, were established. These cells exhibited distinct gene expression profiles reflecting different functional roles. Specifically, the findings described herein demonstrate that progenitor NP cells residing at the center of the NP give rise to mature NP cells that reside at the periphery. Histological findings provide evidence that this transition from progenitor to mature NP cells occurs progressively during postnatal growth, and may ultimately lead to the progenitor population being exhausted. A recent study described the emergence of notochord-derived, chondrocyte-like cells in the postnatal mouse disc from 16 months-of-age (Mohanty, S, et al. Aging Cell 2019; 18:e13006) These cells, termed “late stage” NP cells, were observed to fuse into nest-like structures and were associated with pathological aging. These cells appear to be distinct from the mature NP cells identified in the current study, which expressed anabolic markers typically associated with the healthy NP ECM, including aggrecan, collagens II and VI, and sox (Le Maitre, et al. Biochem Soc Trans 2007; 35:652-655). The results described herein show that these cells are primarily responsible for elaborating the proteoglycan-rich ECM of the healthy NP, and may represent an intermediate phenotype that transitions towards an aged or pathological state later in life.

In the current study, pathway analysis of scRNA-seq results identified ECM-receptor interaction, protein digestion and absorption, and focal adhesion signaling as top pathways enriched in mature NP cells, which is consistent with an important role for these cells in ECM elaboration. Additionally, the TGF-β and PI3K/Akt signaling pathways were significantly enriched in mature versus progenitor NP cells. Embryonically. TGF-β signaling has been shown to be important for formation of both the NP and AF (Baffi, M O, et al. Dev Biol 2004; 276:124-142; and Chen, S, et al. Osteoarthritis and cartilage 2019; 27:1109-1117). In a prior study, it was shown that TGF-β expression is elevated in mouse NP cells at the onset of postnatal growth concomitantly with elevated ECM expression (Peck, S H, et al. Scientific reports 2017; 7:10504). In vitro, adult NP cells treated with exogenous TGF-β increase production of healthy ECM components (Smith, L J, et al. Eur Cell Mater 2011; 22:291-301). In the current study, elevated TGF-β signaling in mature NP cells, specifically, is consistent with a central role in ECM elaboration and organization. PI3K/Akt signaling is a complex pathway involving more than 150 proteins, is activated by several growth factors, cytokines, and mechanical stimuli, and performs functions in many cellular processes essential for homeostasis, including regulation of cell proliferation, survival, inflammation, metabolism, and apoptosis (Sun, K, et al. Osteoarthritis and cartilage 2020; 28:400-409; and Hemmings, B A, and Restuccia, D F, Cold Spring Harb Perspect Biol 2015; 7). While PI3K/Akt signaling has not been studied in the context of disc development, though inhibition of PI3K/Akt signaling in rat NP cells was found to decrease both proliferation and ECM expression (Xiao, Q, et al. Biomed Res Int 2021; 2021:9941253).

Evidence from prior studies suggests that secreted factors from progenitor (notochordal) NP cells regulates ECM production in mature (chondrocyte-like) NP cells (Aguiar, D J, et al. Exp Cell Res 1999; 246:129-137). In the current study, analysis of cell-cell interactions identified two secreted factors. Ptn and Spp1, that may signal from progenitor to mature NP cells. Pleiotrophin is a developmentally-regulated heparin-binding protein abundant in fetal, but not adult articular cartilage, which has been shown to inhibit proliferation and stimulate glycosaminoglycan synthesis in mature chondrocytes (Tapp, H, et al. Matrix Biol 1999; 18:543-556). Interestingly, a prior study found that Ptn is expressed at higher levels in NP cells compared to articular chondrocytes (Lee, C R, et al. Eur Spine J 2007; 16:2174-2185). It is possible that secretion of Ptn by progenitor NP cells stimulates proteoglycan production by mature stage NP cells. Osteopontin is a secreted, integrin-binding, glycosylated phosphoprotein important in biomineralization and pathological cartilage remodeling (Bai, R J, et al. Front Endocrinol (Lausanne) 2022; 13:1012508). Additional roles include regulating cell migration and adhesion, and ECM organization (Denhardt, D T, et al. Ann N Y Acad Sci 1995; 760:127-142), with Spp1 knockout mice exhibiting aberrant wound healing (Liaw, L, et al. J Clin Invest 1998; 101:1468-1478). Interestingly. Spp1 was previously shown to be expressed in the notochord (Thayer, J M, et al. J Exp Zool 1995; 272:240-244; and Zhou, T, et al. Adv Sci (Weinh) 2023; 10:e2206296), and a study used single cell sequencing analysis to identify Spp1 as playing an important role in intercellular crosstalk during early human disc formation (Zhou, T, et al. Adv Sci (Weinh) 2023; 10:e2206296).

Two surface markers, Cd9 and Cd109, were also identified that were significantly elevated in their expression in mature versus progenitor NP cells. Two recent studies used proteomics to identify Cd109 as a novel NP-specific marker in both mice and humans (Kudelko, M, et al. Matrix Biol Plus 2021; 12:100082; and Tam. V. et al. Elife 2020; 9). Cd109 is a glycosylphosphatidylinositol-anchored glycoprotein (Lin, M, et al. Blood 2002; 99:1683-1691), and while its function in the disc is unknown, in other tissues such as skin it functions as a negative regulator of TGF-β signaling (Finnson, K W, et al. FASEB J 2006; 20:1525-1527). No previous studies have examined the expression or role of Cd9 in the NP. Interestingly, immunofluorescent staining showed that Cd109 protein expression was relatively homogenous across tdTomato+ NP cells, while Cd9 expression was clearly confined to a subset of NP cells that were localized to the peripheral zone. Further, the relative number of Cd9+ cells increased with age, and was co-incident with the emerging halo of glycosaminoglycan-rich ECM. These findings suggest that Cd9 may be a more faithful marker of mature NP cells, specifically, relative to Cd109. As further evidence that these cells are associated with the healthy NP ECM, a preclinical goat model was used to show that the number of Cd9+ NP cells comprise the majority of total cells in healthy adult goat discs, but are significantly diminished with disc degeneration. Cd9, a member of the tetraspanin family of molecules, is a cell surface glycoprotein expressed by many different cell types, and which has diverse roles including in growth, differentiation, cell adhesion, motility and regulation of inflammation (Brosseau, C, et al. Front Immunol 2018; 9:2316; and Powner, D, et al. Biochem Soc Trans 2011; 39:563-567). Interestingly, in other cell types including immune, lung and endothelial cells, Cd9 interacts with integrins, reflecting an important role in cell adhesion and interactions with the ECM (Powner, D, et al. Biochem Soc Trans 2011; 39:563-567). In keratinocytes, Cd9, through interactions with E-cadherin, has been shown to activate PI3K-Akt signaling (enriched in mature NP cells) (Jiang, X, et al Biochim Biophys Acta Mol Cell Res 2020; 1867:118574). Notably, Cd9 is also marker of extracellular vesicles (Brosseau, C, et al. Front Immunol 2018; 9:2316).

It is worth noting that the sequenced cell population was heterogenous, and included both mesenchymal and hematopoietic cells. This heterogeneity was surprising given in situ imaging that showed tdTomato expression confined to the NP region. Similar heterogeneity was reported previously for cells enriched from other Cre-driven tdTomato reporter mice (Yu, W, et al. J Clin Invest 2021; 131). As notochordal cells are highly secretory, it is possible that tdTomato protein is transferred to adjacent cell populations and is detectable by the laser during cell sorting even when present at low levels. This contention is supported by previous in vitro co-culture studies of progenitor and differentiated cells, where chemical fluorescent markers are readily transferred from labeled to adjacent unlabeled cells (Kim, M, et al. Proc Natl Acad Sci USA 2019; 116:1569-1578).

In conclusion, described herein are the fate and function of different cell types that comprise the postnatal NP. Specifically, the results showed that progenitor NP cells give rise to mature NP cells that are characterized by higher expression of healthy ECM genes and the cell surface marker Cd9.

All publications, patents, and patent applications cited herein are hereby incorporated herein by reference in their entirety.

1. A cellular composition, comprising:
a protectant, and
isolated mammalian nucleus pulposus cells, wherein the isolated mammalian nucleus of pulposus cells have an extracellular matrix (ECM)-generating phenotype, wherein at least 90% of cells in the isolated mammalian nucleus pulposus cells express CD9, and wherein at least 90% of cells in the isolated mammalian nucleus pulposus cells express CD109, wherein the isolated mammalian nucleus pulposus cells have an increased expression of CD9, CD109, or both CD9 and CD109 compared to the expression of CD9, CD109, or both CD9 and CD109 in premature nucleus pulposus cells.

2.-4. (canceled)

5. The cellular composition of claim 1, wherein the isolated mammalian nucleus pulposus cells have an increased expression of nucleus pulposus-specific ECM genes compared to the expression of the same genes in premature nucleus pulposus cells.

6. The cellular composition of claim 1, wherein the isolated mammalian nucleus pulposus cells express one or more genes selected from the group consisting of ACAN, COL2A1, and COL6A3.

7. The cellular composition of claim 6, wherein the isolated mammalian nucleus pulposus cells have an increased expression of ACAN, COL2A1, and COL6A3 compared to the expression of ACAN, COL2A1, and COL6A3 in premature nucleus pulposus cells.

8. The cellular composition claim 1, wherein the isolated mammalian nucleus pulposus cells comprise or consist essentially of mature mammalian nucleus pulposus cells.

9. The cellular composition of claim 8, wherein the mature mammalian nucleus pulposus cells have an increased expression of nucleus pulposus-specific ECM genes relative to the expression of the same genes in premature nucleus pulposus cells.

10. The cellular composition of claim 1, wherein the protectant comprises a hydrogel.

11. The cellular composition of claim 1, wherein the protectant is at least 50% volume to volume of the isolated mammalian nucleus pulposus cells.

12. The cellular composition of claim 1, wherein the isolated mammalian nucleus pulposus cells are derived from notochord-derived nucleus pulposus cells.

13. A cellular composition comprising:
a population of mammalian nucleus pulposus cells having an extracellular matrix (ECM)-generating phenotype, wherein the mammalian nucleus pulposus cells:
(i) express CD9 and have an anti-CD9 antibody bound to at least a portion of the cells in the population, and/or
(ii) express CD109 and have an anti-CD109 antibody bound to at least a portion of the cells in the population, and
a protectant, wherein the population of isolated mammalian nucleus pulposus cells have an increased expression of CD9, CD109, or both CD9 and CD109 compared to the expression of CD9, CD109, or both CD9 and CD109 in premature nucleus pulposus cells.

14. (canceled)

15. The cellular composition of claim 13, wherein the population of isolated mammalian nucleus pulposus cells have an increased expression of nucleus pulposus-specific ECM genes compared to the expression of same genes in premature nucleus pulposus cells.

16. The cellular composition of claim 13, wherein the population of isolated mammalian nucleus pulposus cells express one or more genes selected from the group consisting of ACAN, COL2A1, and COL6A3.

17. The cellular composition of claim 16, wherein the population of isolated mammalian nucleus pulposus cells have an increased expression of ACAN, COL2A1, and COL6A3 compared to the expression of ACAN, COL2A1, and COL6A3 in premature nucleus pulposus cells.

18. The cellular composition of claim 13, wherein the population of isolated mammalian nucleus pulposus cells comprise mature mammalian nucleus pulposus cells.

19. The cellular composition of claim 18, wherein the mature mammalian nucleus pulposus cells have an increased expression of nucleus pulposus-specific ECM genes compared to the expression of the same genes in premature nucleus pulposus cells.

20. The cellular composition of any of claim 13, wherein the cellular composition further comprises mesenchymal stem cells and/or aggrecan.

21. A method of treating a subject, the method comprising administering a cellular composition to the subject, the cellular composition, comprising: a protectant, and isolated mammalian nucleus pulposus cells, wherein the isolated mammalian nucleus of pulposus cells of nucleus pulposus cells have an extracellular matrix (ECM)-generating phenotype, wherein the mammalian nucleus pulposus cells: (i) express CD9 and have an anti-CD9 antibody bound to at least a portion of the cells in the population, and/or (ii) express CD109 and have an anti-CD109 antibody bound to at least a portion of the cells in the population, wherein the population of isolated mammalian nucleus pulposus cells have an increased expression of CD9, CD109, or both CD9 and CD109 compared to the expression of CD9, CD109, or both CD9 and CD109 in premature nucleus pulposus cells.

22. (canceled)

23. (canceled)

24. The method of claims 21, wherein the subject has back pain.

25. The method of claim 21, wherein the administering comprises administering to an intervertebral disc of the subject.

26. The method of claim 21, wherein the composition comprises isolated allogeneic nucleus pulposus cells.

27.-35. (canceled)
